(19) United States
(12) Patent Application Publication
(10) Pub. No.: US 2023/0312510 A1
(43) Pub. Date: Oct. 5, 2023

(54) COMPOUNDS AND METHODS FOR TREATING NEURODEGENERATIVE DISEASES

(71) Applicant: The McLean Hospital Corporation, Belmont, MA (US)

(72) Inventors: Kwang-Soo Kim, Lexington, MA (US); Woori Kim, Belmont, MA (US)

(21) Appl. No.: 18/013,155

(22) PCT Filed: Jul. 7, 2021
(86) PCT No.: PCT/US2021/040637

§ 371 Date: Dec. 27, 2022

Related U.S. Application Data
(60) Provisional application No. 63/048,829, filed on Jul. 7, 2020.

(51) Int. Cl. C07D 401/12; C07D 401/14; C07D 409/14; A61P 25/28

(52) U.S. Cl. CPC C07D 401/12; C07D 401/14; C07D 409/14; A61P 25/28

(57) ABSTRACT

The present application provides compounds and methods, e.g., for activating Nurr 1 and for treating diseases and conditions in which Nurr 1 is implicated.

### CLAIM OF PRIORITY

This application claims priority to U.S. Patent Application Ser. No. 63/048,829, filed on Jul. 7, 2020, the entire contents of which are hereby incorporated by reference.

### TECHNICAL FIELD

This invention relates to quinoline compounds, and in particular to compounds useful for treating neurodegenerative diseases.

### BACKGROUND

There are numerous deadly diseases affecting current human population. For example, neurodegenerative diseases affect a significant segment of population, especially the elderly. Parkinson's disease (“PD”) is a neurodegenerative disorder that affects approximately 6.1 million people world-wide with an estimated socioeconomic burden of more than $52 billion.

### SUMMARY

In one general aspect, the present disclosure provides a compound selected from any one of the following compounds:

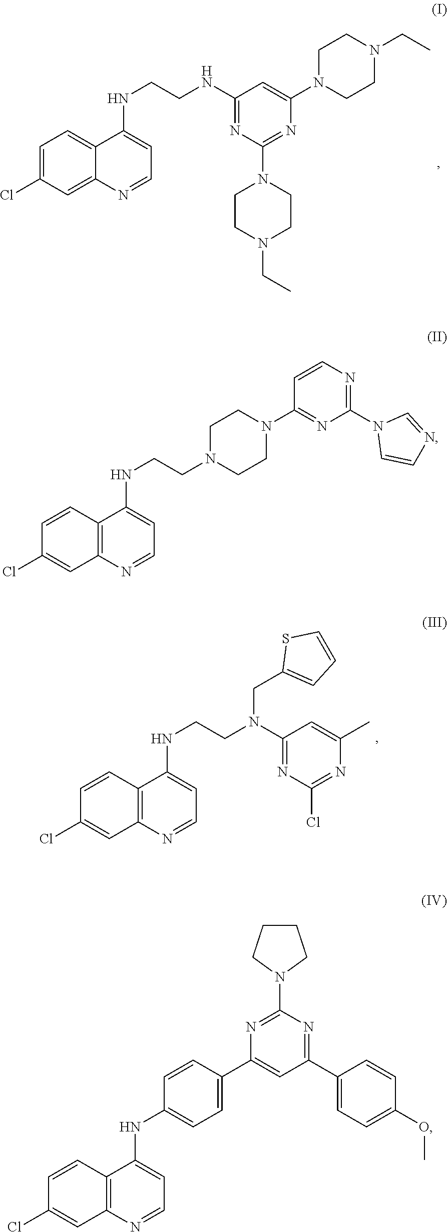 [SMILES: CCN1CCN(c2cc(NCCNc3ccnc4cc(Cl)ccc34)nc(N3CCN(CC)CC3)n2)CC1 ; Clc1ccc2c(NCCN3CCN(c4ccnc(-n5ccnc5)n4)CC3)ccnc2c1 ; Cc1cc(N(CCNc2ccnc3cc(Cl)ccc23)Cc2cccs2)nc(Cl)n1 ; COc1ccc(-c2cc(-c3ccc(Nc4ccnc5cc(Cl)ccc45)cc3)nc(N3CCCC3)n2)cc1]

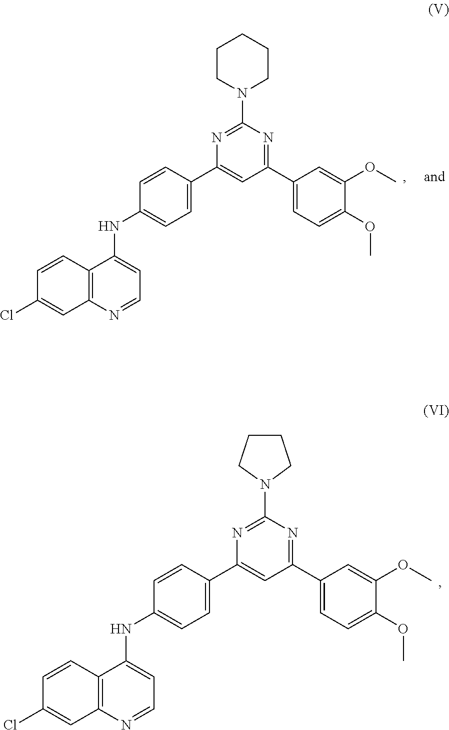 [SMILES: COc1ccc(-c2cc(-c3ccc(Nc4ccnc5cc(Cl)ccc45)cc3)nc(N3CCCCC3)n2)cc1OC ; COc1ccc(-c2cc(-c3ccc(Nc4ccnc5cc(Cl)ccc45)cc3)nc(N3CCCC3)n2)cc1OC]

or a pharmaceutically acceptable salt thereof.

In some embodiments, the compound has Formula (I):

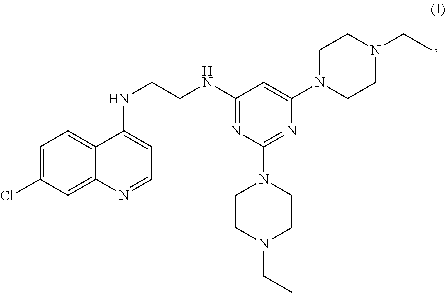 [SMILES: CCN1CCN(c2cc(NCCNc3ccnc4cc(Cl)ccc34)nc(N3CCN(CC)CC3)n2)CC1 ; ","]

or a pharmaceutically acceptable salt thereof.

In some embodiments, the compound has Formula (II):

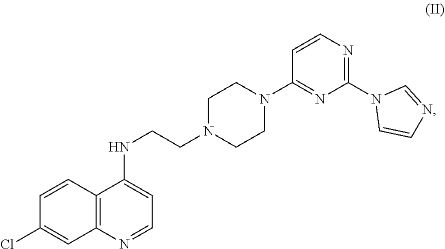 [SMILES: Clc1ccc2c(NCCN3CCN(c4ccnc(-n5ccnc5)n4)CC3)ccnc2c1]

or a pharmaceutically acceptable salt thereof.

In some embodiments, the compound has Formula (III):

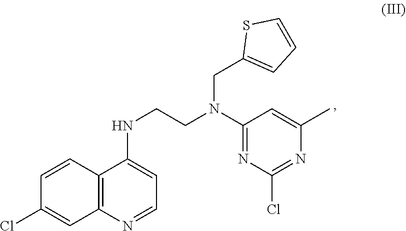 [SMILES: Cc1cc(N(CCNc2ccnc3cc(Cl)ccc23)Cc2cccs2)nc(Cl)n1 ; ","]

or a pharmaceutically acceptable salt thereof.

In some embodiments, the compound has Formula (IV):

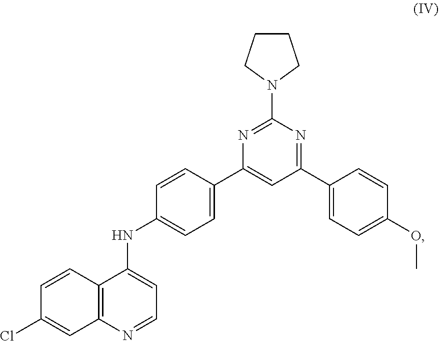 [SMILES: COc1ccc(-c2cc(-c3ccc(Nc4ccnc5cc(Cl)ccc45)cc3)nc(N3CCCC3)n2)cc1]

or a pharmaceutically acceptable salt thereof.

In some embodiments, the compound has Formula (IV):

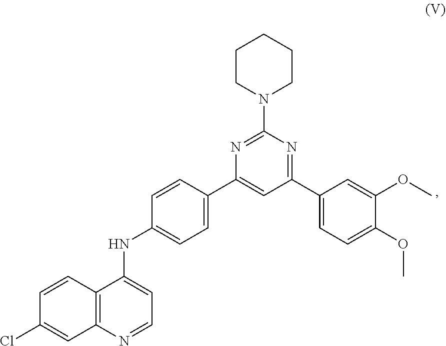 [SMILES: COc1ccc(-c2cc(-c3ccc(Nc4ccnc5cc(Cl)ccc45)cc3)nc(N3CCCCC3)n2)cc1OC ; ","]

or a pharmaceutically acceptable salt thereof.

In some embodiments, the compound has Formula (IV):

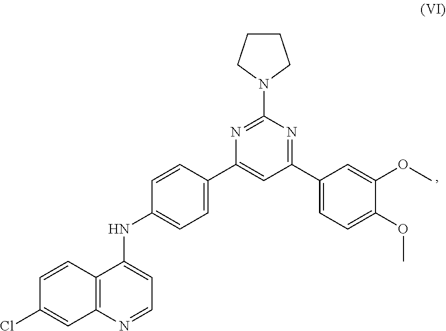 [SMILES: COc1ccc(-c2cc(-c3ccc(Nc4ccnc5cc(Cl)ccc45)cc3)nc(N3CCCC3)n2)cc1OC ; ","]

or a pharmaceutically acceptable salt thereof.

In another general aspect, the present disclosure provides a pharmaceutical composition comprising a compound of Formula (I), or a pharmaceutically acceptable salt thereof, and a pharmaceutically acceptable carrier.

In another general aspect, the present disclosure provides a method of modulating Nurr1 activity in a cell, the method comprising contacting the cell with an effective amount of a compound of Formula (I), or a pharmaceutically acceptable salt thereof.

In some embodiments, the modulating of the Nurr1 activity comprises increasing the Nurr1 activity in the cell.

In some embodiments, the method comprises contacting the cell in vivo.

In some embodiments, the method comprises contacting the cell in vitro.

In some embodiments, the method comprises contacting the cell ex vivo.

In another general aspect, the present disclosure provides a method of modulating Nurr1 activity in a cell of a subject, the method comprising administering to the subject an effective amount of a compound of Formula (I), or a pharmaceutically acceptable salt thereof, or a pharmaceutical composition comprising same.

In some embodiments, the method comprises increasing the Nurr1 activity in the cell of the subject.

In another general aspect, the present disclosure provides a method of treating a disease or condition in which decreased Nurr1 activity or Nurr1 hypoactivity contributes to the pathology or symptomology of the disease, the method comprising administering to the subject a therapeutically effective amount of a compound of Formula (I), or a pharmaceutically acceptable salt thereof, or a pharmaceutical composition comprising same.

In some embodiments, the disease or condition is a neurodegenerative disease.

In some embodiments, the neurodegenerative disease is Parkinson's disease.

In some embodiments, the neurodegenerative disease is Alzheimer's disease.

In some embodiments, the method further comprises administering to the subject a second therapeutic agent useful in treating the neurodegenerative disease.

In some embodiments, the disease or condition is inflammation or inflammation-associated disease or condition.

In some embodiments, the method further comprises administering to the subject a second therapeutic agent useful in treating the inflammation or the inflammation-associated disease or condition.

In another general aspect, the present disclosure provides a method of treating an infectious disease or disorder, the method comprising administering to the subject a therapeutically effective amount of a compound of Formula (I), or a pharmaceutically acceptable salt thereof, or a pharmaceutical composition of comprising same.

In some embodiments, the infectious disease is malaria.

In some embodiments, the method further comprises administering to the subject a second therapeutic agent useful in treating the infectious disease or disorder.

In another general aspect, the present disclosure provides a method of inducing differentiation of a stem cell into a dopaminergic neuron, the method comprising contacting the stem cell with a compound of Formula (I), or a pharmaceutically acceptable salt thereof.

In some embodiments, the stem cell is a human embryonic stem cell.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1 contains chemical structures of SPV-94 and chloroquine (CQ).

FIG. 2 contains relative luciferase activities of SPV-94 and previously reported compounds. SPV-94 showed markedly highest transactivity among the selected candidates in SK-N-BE(2)C cells. Each bar indicates Mean±SEM from three independent experiments.

FIG. 3 shows pharmacokinetics of CQ and SPV-94. Intravenous (i.v.) injection of CQ or SPV-94 in rats (5 mg/kg) showed that SPV-94 has slower penetration into the brain than CQ. Rats (n=6) were killed at 5 min and 1 hr after i.v. administration, and brain and plasma were collected for blood-brain barrier (BBB) penetration analysis. The concentrations of each compound in plasma (A) and brain (B) were determined by LC-MS/MS (liquid chromatography-tandem mass spectrometry). The brain/plasma ratio (B/P ratio) (C) is calculated at each time point.

FIG. 4B shows interaction of CQ or SPV-94 with Nurr1-LBD. Competition of CQ or SPV-94 with [3H]-CQ for binding to Nurr1-LBD was assessed by incubating unlabeled competitors with 1,000 nM of [³H]-CQ and 0.2 μM of Nurr1-LBD. The estimated half maximal inhibitory concentration (IC₅₀) of CQ and SPV-94 is 1 μM and 50 nM, respectively.

FIG. 4C shows that CQ and SPV-94 enhanced transcriptional activities of Nurr1-LBD in a dose dependent manner in SK-N-BE(2)C cells. SPV-94 reached its maximal efficiency at 20 μM, which is 5-fold lower than CQ. The half maximal effective concentrations (EC₅₀) of CQ and SPV-94 are 50 μM and 10 μM, respectively.

FIG. 4D shows that CQ and SPV-94 enhanced transcriptional activities of full-length Nurr1 in a dose dependent manner in SK-N-BE(2)C cells. SPV-94 reached its maximal efficiency at 20 μM, which is 5-fold lower than CQ. The half maximal effective concentrations (EC50) of CQ and SPV-94 are 50 μM and 10 μM, respectively.

FIG. 5A shown Nurr1 transactivation and protective effects of CQ and SPV-94 in MN9D cell line. CQ and SPV-94 enhanced transcriptional activities of both Nurr1 -LBD and full-length Nurr1 in a dose dependent manner in MN9D.

FIG. 5B shows Nurr1 transactivation and protective effects of CQ and SPV-94 in N27-A cell line. CQ and SPV-94 enhanced transcriptional activities of both Nurr1-LBD and full-length Nurr1 in a dose dependent manner in N27-A cells.

FIG. 5C shows that Both CQ and SPV-94 dose-dependently increased cell viability in MTT assay and reduced cytotoxicity in LDH assay compared to 1 mM of MPP+-treated condition in N27-A cells. *p<0.05, **p<0.01, ***p<0.001 compared to 0 μM, Student's t-test.

FIG. 6 shows that point mutations on potential binding residues of Nurr1-LBD (S441, 1573, 1588, K590, L593, D594, T595, L596 or F598) failed to induce CQ (100 μM) or SPV-94 (20 μM) induced Nurr1 transactivation in SK-N-BE(2)C cells. ***p <0.001 compared to CQ or SPV-94 treated wild-type (WT), one-way ANOVA, Tukey's post-hoc test.

FIG. 7A shows protective effects of CQ and SPV-94 against MPP+-induced oxidative stress in MN9D cells. Cell viability and cytotoxicity were measured by MTT (3-(4,5-dimethylthiazol-2-yl)-2,5-diphenyltetrazolium bromide) reduction assay. Both CQ and SPV-94 dose-dependently increased cell viability and reduced cytotoxicity compared to 500 μM of MPP+-treated condition in MN9D cells. *p<0.05, **p<0.01, ***p<0.001 compared to 0 μM, Student's t-test.

FIG. 7B shows protective effects of CQ and SPV-94 against MPP+-induced oxidative stress in MN9D cells. Cell viability and cytotoxicity were measured by lactate dehydrogenase (LDH) release assay. Both CQ and SPV-94 dose-dependently increased cell viability and reduced cytotoxicity compared to 500 μM of MPP+-treated condition in MN9D cells. *p<0.05, **p<0.01, ***p<0.001 compared to 0 μM, Student's t-test.

FIGS. 7C-7F show cell viability analyzed by MTT reduction (7C and 7D) and cytotoxicity measured using LDH release (7E and 7F) showed that Nurr1 overexpression (OE) potentiated protective effects of CQ (100 μM) and SPV-94 (1 μM) against MPP⁺-induced toxicity compared to Mock control in MN9D cells (7C and 7E). However, Nurr1 knockdown (KD) diminished the protective effects of CQ and SPV-94 both in normal and MPP⁺-induced toxic conditions. **p<0.01, ***p<0.001 compared to vehicle (VEH) treatment under Mock or Scramble conditions; ##p<0.01, ###p<0.001 compared between each treatment group, one-way ANOVA, Tukey's post-hoc test.

FIGS. 7G-7H show that Nurr1 protein expression levels significantly increased in OE with Nurr1 -LBD transfection (7G) or decreased in KD with shNurr1 transfection (7H) in MN9D cells. **p<0.01, ***p<0.001 compared to Mock or scramble (Scr.) controls, Student's t-test.

FIGS. 8A-8D show dopaminergic (DAergic) gene expressions in the absence or presence of 6-OHDA (20 μM) in mouse embryonic ventral mesencephalic (mVM) primary neurons derived from embryonic day 12.5 (E12.5). CQ (20 μM) (8A) and SPV-94 (0.5 μM) (8C) significantly upregulated mRNA expression levels of DAergic genes such as tyrosine hydroxylase (TH), dopamine transporter (DAT), aromatic L-amino acid decarboxylase (AADC), vesicular monoamine transporter 2 (VMAT2), c-Ret and paired like homeodomain 3 (Pitx3) compare to vehicle treated group. Furthermore, CQ and SPV-94 resulted in significant recovery of downregulated DAergic gene expressions induced by 6-01-IDA toxicity. These effects by CQ and SPV-94 disappeared in Nurr1 knockdown (1(D) condition (8B and 8D). *p<0.05, **p<0.01, ***p<0.001 compared to vehicle treatment in Scramble condition (Control); #p<0.05, ##p<0.01, ###p<0.001 compared between each treatment group, one-way ANOVA, Tukey's post-hoc test. Each bar was transformed as a fold relative to control. Error bars represent SEM.

FIGS. 9A-9J show BV2 cells (9A) and mouse bone marrow-derived primary macrophages (mBMMs) (9B) were treated with CQ or SPV-94 in the presence of LPS (1 μg/ml) which activates inflammation via toll-like receptor 4 (TLR4). mRNA expression of tumor necrosis factor alpha (TNFα) was determined by real-time PCR. At 1 μM concentration, CQ and SPV-94 robustly suppressed TNFα expression down by 35.53% and 20.67% respectively, compared to LPS only. *p<0.05, ***p<0.001 compared to LPS only, Student's t-test. (9C-9J) Immune suppression by CQ and SPV-94 against LPS (1μg/ml) or poly(I:C) (1 μg/ml) in mBMMs. (9C-9F) Four pro-inflammatory genes such as TNFα, iNOS, IL-10 and IL-6 were highly upregulated by incubating cells with LPS or poly(I:C) for overnight. CQ (10 μM) treatment significantly suppressed LPS- or poly(I:C)-induced pro-inflammatory gene expressions. (9G-9J) Same as CQ, but even at 10 times lower concentration, SPV-94 dramatically suppressed all four pro-inflammatory gene expressions. **p<0.01, ***p<0.001 compared to LPS or poly(I:C) only, one-way ANOVA, Tukey's post-hoc test.

FIGS. 10A-10C show HeLa cells incubated in starvation medium (Earle's Balanced Salt Solution, EBSS) containing bafilomycin A1 (BafA1, 10 nM), CQ (20 μM), or SPV-94 (1 μM) for 0-4 hrs. To limit basal autophagy, cells were incubated in fresh growth medium for 1 hr before starvation. Samples were analyzed by Western blot using autophagic flux markers LC3B and p62 (A) and its expression levels were quantified (10B and 10C). BafA1 and CQ treatments induced autophagy initiation but inhibited autophagy process termination. On the other hand, SPV-94 initiated and also terminated autophagy process resulting in significant p62 degradation by time. *p<0.05, **p<0.01, ***p<0.001; #p<0.05, ##p<0.01, ###p<0.001 compared to VEH, one-way ANOVA, Dunnett's multiple comparisons.

FIGS. 10D-10G show N27-A cells that were incubated in starvation medium containing BafA1 (10 nM), CQ (20 μM), or SPV-94 (1 μM) for 0-4 hrs. LC3B, p62 and Nurr1 expression levels determined by Western blot (D) were quantified. Similar in HeLa cells, autophagy was successfully terminated by SPV-94 treatment but not by BafA1 or CQ treatments (10E and 10F). Interestingly, basal Nurr1 level was significantly higher in CQ or SPV-94 treated groups compared to VEH group. Throughout autophagy process, Nurr1 expression level was gradually decreased, but due to its higher initial expression by CQ and SPV-94, Nurr1 expression remained significantly higher than in VEH group. *p<0.05, **p<0.01, ***p<0.001; #p<0.05, ##p<0.01, ###p<0.001 compared to VEH, one-way ANOVA, Tukey's multiple comparisons.

FIG. 11A contains schematic representation of CQ and SPV-94 administrations to MPTP-treated mice. CQ (40 mg/kg) and SPV-94 (5 mg/kg) were administered starting from MPTP injection and continued for 16 days. Sub-chronic MPTP regimen (30 mg/kg/day, 5 days) was introduced. L-DOPA administration (50 mg/kg/day) was introduced for 16 days, along with CQ and SPV-94 treatments.

FIG. 11B contains line plots showing that body weight changes showed significant reduction after day 2 in MPTP treated group compared to vehicle-treated group (VEH). L-DOPA and CQ treated groups regained body weight after day 8, and SPV-94 treated group restored it even earlier, after day 6. *p<0.05, **p<0.01, ***p<0.001 compared to VEH, two-way ANOVA, Sidak's post-hoc test.

FIGS. 11C-11E show that sub-chronic treatments of L-DOPA, CQ and SPV-94 significantly improved motor deficits on the rotarod latency to fall (11C), reduced time to traverse on a pole (11D), and recovered rearing numbers in the cylinder test (11E). **p<0.01, ***p<0.001 compared to vehicle-treated group (VEH); #p<0.05, ##p<0.01 compared.

FIGS. 11F-11G show CQ and SPV-94 treatments significantly recovered olfaction. Both CQ and SPV-94 significantly increased duration to stay in the old bedding (familiar odor) compared to new bedding (non-familiar odor) in olfactory discrimination test. L-DOPA, on the other hand, failed to restore olfaction (11F). L-DOPA treated group showed hyperactivity indicated as increased velocity during olfactory discrimination (11G). *p<0.05, **p<0.01, ***p<0.001, one-way ANOVA, Tukey's post-hoc test; n>7 per group.

FIG. 11H contains line plot showing that chronic administration of L-DOPA developed dyskinesia (LID, L-DOPA induced dyskinesia) in the abnormal involuntary movements (AIMs) test, but neither CQ nor SPV-94 did not trigger dyskinesia.

FIGS. 12A-12E show motor and non-motor behaviors assessed at chronic stages. Motor deficits induced by MPTP treatment retained until day 15, which is 10 days after the last injection. Chronic treatments of L-DOPA, CQ and SPV-94 improved latency to fall on the rotarod on day 15 (12A). Otherwise, MPTP-induced motor impairments tended to be diminished in the pole test and cylinder test at the chronic stage (12B and 12C). *p<0.05 compared to vehicle-treated group (VEH); #p<0.05, ###p<0.001 compared to MPTP treated group, one-way ANOVA, Tukey's post-hoc test; n>7 per group. Impaired olfaction maintained until day 14, and chronic treatments of CQ and SPV-94 but not L-DOPA significantly recovered olfaction (12D), without affecting mobility (12E). *p<0.05, **p<0.01, ***p<0.001, one-way ANOVA, Tukey's post-hoc test; n>7 per group.

FIGS. 13A-13D show that TH immunoreactivity showed that CQ and SPV-94 increased TH+ DAergic neurons in the striatum (STR), substantia nigra pars compacta (SNpc) and olfactory bulb (OB). Scale bars indicate 500 μM (13A). Quantitative analysis of TH+ neurons by counting and densitometry revealed that CQ and SPV-94 treatments significantly restored DAergic neurons in the STR (13B), SNpc (13C) and OB (13D). Meanwhile, L-DOPA treatment did not show protective effect on the TH+DAergic neurons. **p<0.01, ***p<0.001 compared to VEH; #p<0.05, ##p<0.01, ###p<0.001 compared to MPTP treated group, one-way ANOVA, Tukey's post-hoc test; n>7 per group.

FIGS. 13E-13G show that Iba-1 immunoreactive cells increased in MPTP treated group representing increased number of activated microglia both in the STR and SNpc. Scale bars indicate 500 μM (13E). Notably, quantitative data showed that CQ and SPV-94 treatments significantly reduced numbers of Iba-1+ microglia both in the STR (13F) and SNpc (13G), indicating suppression of microglial activation. L-DOPA failed to suppress microglial activation. ***p<0.001 compared to VEH; ##p<0.01, ###p<0.001 compared to MPTP treated group, one-way ANOVA, Tukey's post-hoc test; n>7 per group.

FIGS. 14A-14B show Nurr1 immunoreactivity in the SNpc (14A) showed that CQ and SPV-94 treatments significantly retained Nurr1-immunoreactive cells in the SNpc, otherwise, L-DOPA treatment failed to protect Nurr1 expressions (14B). Scale bar indicates 500 μM. ***p<0.001 compared to VEH; ###p<0.001 compared to MPTP treated group, one-way ANOVA, Tukey's post-hoc test; n>7 per group.

FIG. 15 shows that glial fibrillary acidic protein (GFAP) immunoreactivity in the STR (A) exhibited that CQ and SPV-94 treatments reduced number of activated astrocytes compared to MPTP group, while L-DOPA did not. Scale bar indicates 500 μm.

FIG. 16 contains a table providing cage-side observations of male mice treated with compound of Formula (II) and the compound of comparative example.

FIG. 17 contains a table providing cage-side observations of female mice treated with compound of Formula (II) and the compound of comparative example.

### DETAILED DESCRIPTION

In some embodiments, the present disclosure provides a compound selected from any one of the following compounds:

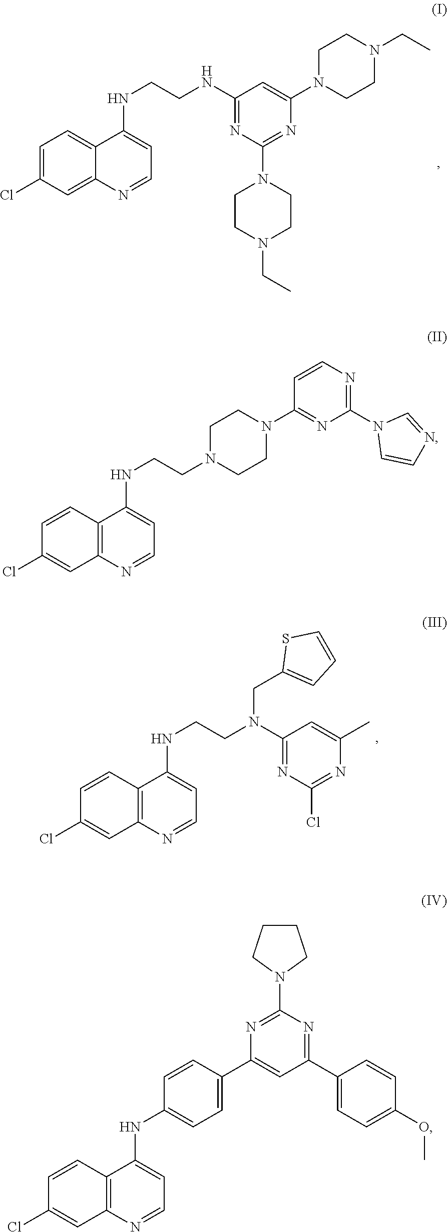 [SMILES: CCN1CCN(c2cc(NCCNc3ccnc4cc(Cl)ccc34)nc(N3CCN(CC)CC3)n2)CC1 ; Clc1ccc2c(NCCN3CCN(c4ccnc(-n5ccnc5)n4)CC3)ccnc2c1 ; Cc1cc(N(CCNc2ccnc3cc(Cl)ccc23)Cc2cccs2)nc(Cl)n1 ; COc1ccc(-c2cc(-c3ccc(Nc4ccnc5cc(Cl)ccc45)cc3)nc(N3CCCC3)n2)cc1]

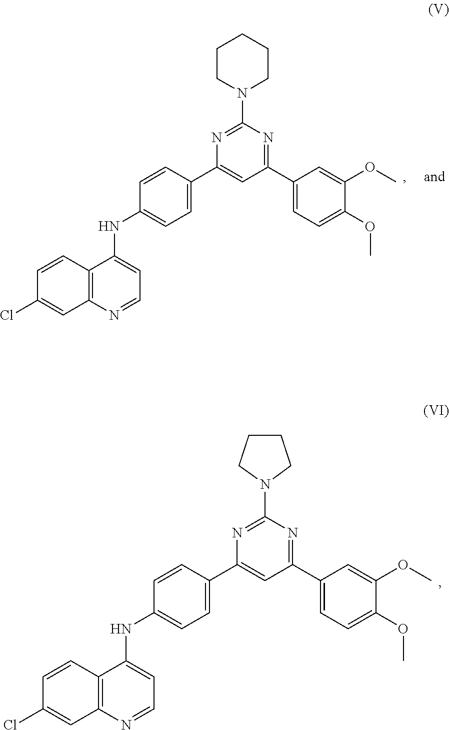 [SMILES: COc1ccc(-c2cc(-c3ccc(Nc4ccnc5cc(Cl)ccc45)cc3)nc(N3CCCCC3)n2)cc1OC ; COc1ccc(-c2cc(-c3ccc(Nc4ccnc5cc(Cl)ccc45)cc3)nc(N3CCCC3)n2)cc1OC]

or a pharmaceutically acceptable salt thereof.

In some embodiments, the present disclosure provides a compound of Formula (I):

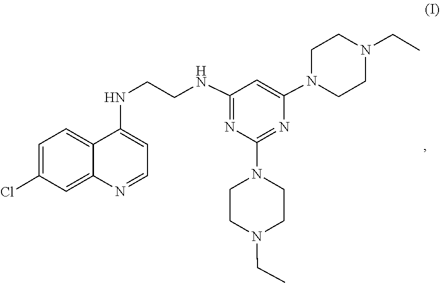 [SMILES: CCN1CCN(c2cc(NCCNc3ccnc4cc(Cl)ccc34)nc(N3CCN(CC)CC3)n2)CC1]

or a pharmaceutically acceptable salt thereof.

In some embodiments, the present disclosure provides a compound of Formula (II):

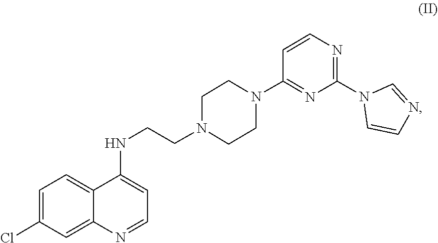 [SMILES: Clc1ccc2c(NCCN3CCN(c4ccnc(-n5ccnc5)n4)CC3)ccnc2c1]

or a pharmaceutically acceptable salt thereof.

In some embodiments, the present disclosure provides a compound of Formula (III):

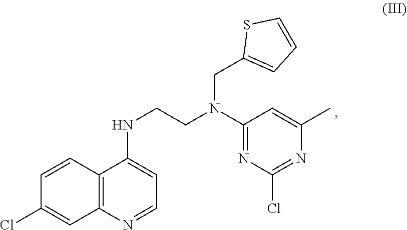 [SMILES: Cc1cc(N(CCNc2ccnc3cc(Cl)ccc23)Cc2cccs2)nc(Cl)n1]

or a pharmaceutically acceptable salt thereof.

In some embodiments, the present disclosure provides a compound of Formula (IV):

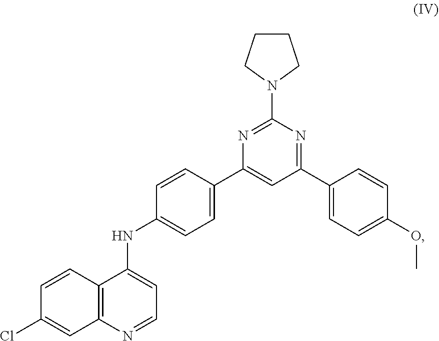 [SMILES: COc1ccc(-c2cc(-c3ccc(Nc4ccnc5cc(Cl)ccc45)cc3)nc(N3CCCC3)n2)cc1]

or a pharmaceutically acceptable salt thereof.

In some embodiments, the present disclosure provides a compound of Formula (IV):

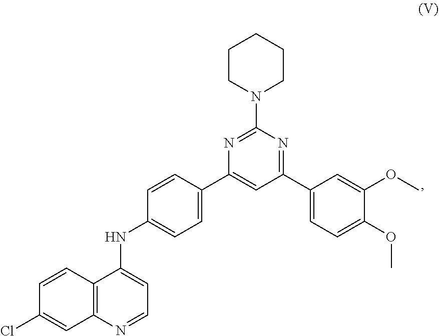 [SMILES: COc1ccc(-c2cc(-c3ccc(Nc4ccnc5cc(Cl)ccc45)cc3)nc(N3CCCCC3)n2)cc1OC]

or a pharmaceutically acceptable salt thereof.

In some embodiments, the present disclosure provides a compound of Formula (IV):

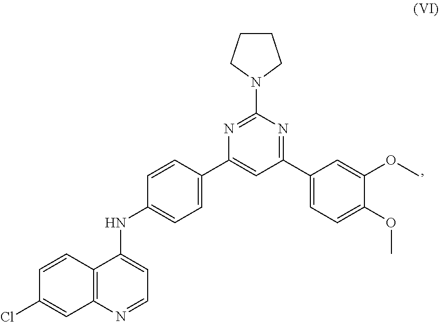 [SMILES: COc1ccc(-c2cc(-c3ccc(Nc4ccnc5cc(Cl)ccc45)cc3)nc(N3CCCC3)n2)cc1OC]

or a pharmaceutically acceptable salt thereof.

Pharmaceutically Acceptable Salts

In some embodiments, a salt of any one of the compounds of the present disclosure (e.g., a compound of Formula (I) or any of the additional therapeutic agents disclosed herein) is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2- sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH-(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

Methods of Making Therapeutic Compounds

The compound of Formula (I), including salts thereof, can be prepared using known organic synthesis techniques and can be synthesized according to any of numerous possible synthetic routes. A person skilled in the art knows how to select and implement appropriate synthetic protocols, and appreciates that the processes described are not the exclusive means by which compounds provided herein may be synthesized, and that a broad repertoire of synthetic organic reactions is available to be potentially employed in synthesizing compounds provided herein.

Suitable synthetic methods of starting materials, intermediates and products may be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry. Reactions, Mechanisms, and Structure, 6^{th} Ed. (Wiley, 2007); Trost et al. (Ed.), Comprehensive Organic Synthesis (Pergamon Press, 1991).

The reactions for preparing the compound of Formula (I) can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in P. G. M. Wuts and T. W. Greene, Protective Groups in Organic Synthesis, 4th Ed., Wiley & Sons, Inc., New York (2006).

Methods of Use

Orphan nuclear receptor Nurr1 (also known as NR4A2) plays a role in development and maintenance of cells such as mDA neurons. Hence, the enhanced activity of Nurr1 is useful for protecting the cells (e.g., neurons) from death such as an inflammation-induced death.

Accordingly, in some embodiments, the present disclosure provides a method of modulating a Nurr1 activity in a cell, the method comprising contacting the cell with an effective amount of a compound of Formula (I), or a pharmaceutically acceptable salt thereof. In some embodiments, the present disclosure provides a method of modulating Nurr1 activity in a cell of a subject, the method comprising administering to the subject an effective amount of a compound of Formula (I), or a pharmaceutically acceptable salt thereof. In one example, the method includes increasing, enhancing, or maintaining the activity of Nurr1 in the cell (e.g., in a dopaminergic neuron). Hence, in some embodiments, the disclosure provides a method of activating Nurr1 in the cell. In some embodiments, the compound of Formula (I) is an agonist of Nurr1 (e.g., the method comprises agonizing Nurr1 in the cell). The cell may be contacted with the compound of Formula (I) in vivo, in vitro, or ex vivo.

In some embodiments, the present disclosure provides a method of treating a disease or condition in which decreased Nurr1 activity or Nurr1 hypoactivity contributes to the pathology or symptomology of the disease, the method comprising administering to the subject a therapeutically effective amount of a compound of Formula (I), or a pharmaceutically acceptable salt thereof. In some embodiments, the disclosure provides a compound of Formula (I) for use in treating a disease or condition in which decreased Nurr1 activity or Nurr1 hypoactivity contributes to the pathology or symptomology of the disease in a subject. In some embodiments, the disclosure provides use of a compound of Formula (I) in the manufacture of a medicament for the treatment of a disease or condition in which decreased Nurr1 activity or Nurr1 hypoactivity contributes to the pathology or symptomology of the disease in a subject.

Numerous publications link neurodegenerative diseases to the decreased Nurr1 activity or Nurr1 hypoactivity, and attest to the neuroprotective effect of Nurr1 activation. These publications demonstrate an association between increased or enhanced activity of Nurr1 or Nurr1 activation and amelioration of symptoms of neurodegenerative diseases. Examples of such publications include US 2009/0226401 to Kim et al., and Moon, M. et al., Nurr1 (NR4A2) regulates Alzheimer's disease-related pathogenesis and cognitive function in the 5XFAD mouse model, Aging Cell, 2019, 18, e12866. Hence, compounds that activate Nurr1 confer neuronal protection and are therefore useful in treating, preventing, or ameliorating symptoms of neurodegenerative diseases.

Neurodegenerative Diseases

Parkinson's disease (“PD”), primarily caused by selective degeneration of midbrain dopamine (“mDA”) neurons, is the most prevalent movement disorder, affecting 1-2% of the global population over the age of 65. Methods of diagnosing subjects as having or being at risk of having Parkinson's Disease are well-known in the art. For example, the presence of one or more of the following symptoms can be used as part of a PD diagnosis: trembling, e.g., an involuntary, rhythmic tremor of one arm or one leg; muscular rigidity, stiffness, or discomfort; general slowness in any of the activities of daily living, e.g., akinesia or bradykinesia; difficulty with walking, balance, or posture; alteration in handwriting; emotional changes; memory loss; speech problems; and difficulty sleeping. Review of a subject's symptoms, activity, medications, concurrent medical problems, or possible toxic exposures can be useful in making a PD diagnosis. In addition, a subject can be tested for the presence or absence of genetic mutations that can indicate an increased likelihood of having Parkinson's Disease. For example, the presence of one or more specific mutations or polymorphisms in the NURR1, alpha-synuclein, parkin, MAPT, DJ-1, PINK1, SNCA, NAT2, or LRRK2 genes can be used to diagnose a subject as having or being at risk of having Parkinson's Disease. See, e.g., U.S. Patent Application Publication Nos. 2003-0119026 and 2005-0186591; Bonifati, Minerva Med. 96:175-186, 2005; and Cookson et al., Curr. Opin. Neurol. 18:706-711, 2005, content of each of which is incorporated herein by reference.

In some embodiments, the present disclosure provides a method of treating, preventing, or ameliorating symptoms of Parkinson's disease, the method comprising administering to a subject in need thereof a therapeutically effective amount of a compound of Formula (I), or a pharmaceutically acceptable salt thereof.

In some embodiments, the present disclosure provides a method of treating, preventing, or ameliorating a symptom of Alzheimer's disease (“AD”), the method comprising administering to a subject in need thereof a therapeutically effective amount of a compound of Formula (I), or a pharmaceutically acceptable salt thereof.

In some embodiments, the compound of Formula (I) is useful in reducing typical AD features, such as deposition of Aβ plaques, neuronal loss, microgliosis, and impairment of adult hippocampal neurogenesis.

Exemplary neurodegenerative disorders that are treatable with the compound of Formula (I) are polyglutamine expansion disorders (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, amyotrophic lateral sclerosis (ALS), ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemia, stroke, Krabbe disease, dementia, Lewy body dementia, multiple sclerosis, multiple system atrophy, Pelizaeus-Merzbacher disease, Pick's disease, primary lateral sclerosis, Refsum's disease, Sandhoff disease, Schilder's disease, spinal cord injury, spinal muscular atrophy (SMA), SteeleRichardson-Olszewski disease, and Tabes dorsalis. Other examples of neurodegenerative disorders that are treatable with the compound of Formula (I) include any disease disorder or condition that affects neuronal homeostasis, e.g., results in the degeneration or loss of neuronal cells. Such diseases include conditions in which the development of the neurons, i.e., motor or brain neurons, is abnormal, as well as conditions in which result in loss of normal neuron function. Examples of such neurodegenerative disorders include Alzheimer's disease and other tauopathies such as frontotemporal dementia, frontotemporal dementia with Parkinsonism, frontotemporal lobe dementia, pallidopontonigral degeneration, progressive supranuclear palsy, multiple system tauopathy, multiple system tauopathy with presenile dementia, Wilhelmsen-Lynch disease, disinhibition-dementia-parkinsonism-amytrophy complex, Pick's disease, or Pick's disease-like dementia, corticobasal degeneration, frontal temporal dementia, Parkinson's disease, Huntington's disease, amyotrophic lateral sclerosis (ALS), multiple sclerosis, Friedreich's ataxia, Lewy body disease, spinal muscular atrophy, and parkinsonism linked to chromosome 17.

Inflammation and Inflammation-Associated Conditions

In another general aspect, the present disclosure provides a method of treating, preventing, or ameliorating a symptom of an inflammation or an inflammation-associated disease or condition, the method comprising administering to the subject a therapeutically effective amount of a compound of Formula (I), or a pharmaceutically acceptable salt thereof, or a pharmaceutical composition comprising same.

Examples of inflammation include reactions of both the specific and non-specific defense systems. A specific defense system reaction is a specific immune system reaction response to an antigen (possibly including an autoantigen). A non-specific defense system reaction is an inflammatory response mediated by leukocytes incapable of immunological memory. Such cells include granulocytes, macrophages, neutrophils and eosinophils. Examples of specific types of inflammation include diffuse inflammation, focal inflammation, croupous inflammation, interstitial inflammation, obliterative inflammation, parenchymatous inflammation, reactive inflammation, specific inflammation, toxic inflammation, and traumatic inflammation.

The compound of Formula (I) inhibits or reduces the expression of pro-inflammatory cytokine genes in primary microglia derived from P1 rat brains. Accordingly, the compound can be used for treating diseases or disorders characterized by elevated levels of pro-inflammatory mediators and/or elevated levels of pro-inflammatory mediator gene expression. Accordingly, the disclosure provides a method for treating a subject suffering from a disease or disorder characterized by elevated levels pro-inflammatory mediators and/or elevated levels of pro-inflammatory mediator gene expression, the method comprising administering a therapeutically effective amount of a compound of Formula (I) to the subject. Exemplary pro-inflammatory mediators include pro-inflammatory cytokines, leukocytes, leukotiens, prostaglandins and other mediators involved in the initiation and maintenance of inflammation. Pro-inflammatory cytokines and inflammation mediators include IL-1-alpha, IL-1-beta, IL-6, IL-8, IL-11, IL-12, IL-17, IL-18, TNF-alpha, leukocyte inhibitory factor (LIF), IFN-gamma, Oncostatin M (OSM), ciliary neurotrophic factor (CNTF), TGF-beta, granulocyte-macrophage colony stimulating factor (GM-CSF), iNOS, and chemokines that chemoattract inflammatory cells. A number of assays for in vivo state of inflammation are known in the art which can be utilized for measuring pro-inflammatory mediator levels. See for example U.S. Pat. Nos. 5,108,899 and 5,550,139, contents of both of which are herein incorporated by reference.

In some embodiments, the disease, disorder, or disease condition characterized by elevated levels of pro-inflammatory cytokines and/or elevated levels of pro-inflammatory cytokine gene expression is an autoimmune disease, neurodegenerative disease, inflammation, an inflammation-associated disorder, a disease characterized by inflammation, or a pathogen or non-pathogen infection.

An autoimmune disease is a disease or disorder wherein the immune system of a subject, e.g., a mammal, mounts a humoral or cellular immune response to the subject's own tissue or to antigenic agents that are not intrinsically harmful to the subject, thereby producing tissue injury in such a subject. Examples of such disorders include, but are not limited to, systemic lupus erythematosus (SLE), mixed connective tissue disease, scleroderma, Sjogren's syndrom, rheumatoid arthritis, and Type I diabetes.

In some embodiments, the inflammation-associated disorder or disease characterized by inflammation is selected from the group consisting of asthma, autoimmune diseases, chronic prostatitis, glomerulonephritis, inflammatory bowl diseases, pelvic inflammatory disease, reperfusion injury, arthritis, silicosis, vasculitis, inflammatory myopathies, hypersensitivities, migraine, psoriasis, gout, artherosclerosis, and any combinations thereof. Exemplary inflammatory diseases include rheumatoid arthritis, inflammatory bowel disease, ulcerative colitis, psoriasis, systemic lupus erythematosus, multiple sclerosis, type 1 diabetes mellitus, multiple sclerosis, psoriasis, vaculitis, and allergic inflammation such as allergic asthma, atopic dermiatitis, and contact hypersensitivity. Other examples of autoimmune-related diseases or disorders include rheumatoid arthritis, multiple sclerosis (MS), systemic lupus erythematosus, Graves' disease (overactive thyroid), Hashimoto's thyroiditis (underactive thyroid), type 1 diabetes mellitus, celiac disease, Crohn's disease and ulcerative colitis, Guillain-Barre syndrome, primary biliary sclerosis/cirrhosis, sclerosing cholangitis, autoimmune hepatitis, Raynaud's phenomenon, scleroderma, Sjogren's syndrome, Goodpasture's syndrome, Wegener's granulomatosis, polymyalgia rheumatica, temporal arteritis/giant cell arteritis, chronic fatigue syndrome CFS), psoriasis, autoimmune Addison's Disease, ankylosing spondylitis, Acute disseminated encephalomyelitis, antiphospholipid antibody syndrome, aplastic anemia, idiopathic thrombocytopenic purpura, Myasthenia gravis, opsoclonus myoclonus syndrome, optic neuritis, Ord's thyroiditis, pemphigus, pernicious anemia, polyarthritis in dogs, Reiter's syndrome, Takayasu's arteritis, warm autoimmune hemolytic anemia, Wegener's granulomatosis, fibromyalgia (FM), autoinflammatory PAPA syndrome, Familial Mediaterranean Fever, familial cold autoinflammatory syndrome, Muckle-Wells syndrome, and the neonatal onset multisystem inflammatory disease.

An anti-inflammation treatment with the compound of Formula (I) aims to prevent or slow down (lessen) an undesired physiological change or disorder, such as the development or progression of the inflammation. Beneficial or desired clinical results include, but are not limited to, alleviation of symptoms, diminishment of extent of disease, stabilized (i.e., not worsening) state of disease, delay or slowing of inflammation disease progression, amelioration or palliation of the disease state, and remission (whether partial or total), whether detectable or undetectable. An anti-inflammation treatment can also mean prolonging survival as compared to expected survival if not receiving treatment. An anti-inflammation treatment can also completely suppress the inflammation response.

One goal of anti-inflammatory treatment is to bring pro-inflammatory mediator levels down to as close to normal as, is safely possible. Accordingly, in one embodiment, level of at least one pro-inflammatory mediator in the subject undergoing treatment is reduced by at least 5%, at least 10%, at least 20%, at least 30%, at least 40%, or at least 50% relative to a reference level. A reference level can be the level of the pro-inflammatory mediator in the subject before onset of treatment regime.

Infectious Diseases

In another general aspect, the present disclosure provides a method of treating an infectious disease or disorder, the method comprising administering to the subject a therapeutically effective amount of a compound of Formula (I), or a pharmaceutically acceptable salt thereof. A parasite causing the infection can be a Plasmodium microorganism. In some embodiments, the infectious disease is malaria. Malaria causes symptoms that typically include fever, tiredness, vomiting, and headaches. The administration of the compound of Formula (I) to a subject ameliorates these symptoms.

Stem Cell Differentiation

In some embodiments, the present disclosure provides a method for causing differentiation of a cell, e.g., a stem cell, into a dopaminergic neuron by contacting the cell with a compound disclosed herein.

Stem cells are unique cell populations that have the ability to divide (self-renew) for indefinite periods of time, and, under the right conditions or signals, to differentiate into the many different cell types that make up an organism. Stem cells derived from the inner cell mass of the blastocyst are known as embryonic stem (ES) cells. Stem cells derived from the primordial germ cells, and which normally develop into mature gametes (eggs and sperm) are known as embryonic germ (EG) cells. Both of these types of stem cells are known as pluripotent cells because of their unique ability to differentiate into derivatives of all three embryonic germ layers (endoderm, mesoderm, and ectoderm).

The pluripotent stem cells can further specialize into another type of multipotent stem cell often derived from adult tissues. Multipotent stem cells are also able to undergo self-renewal and differentiation, but unlike embryonic stem cells, are committed to give rise to cells that have a particular function. Examples of adult stem cells include hematopoietic stem cells (HSC), which can proliferate and differentiate to produce lymphoid and myeloid cell types; bone marrow-derived stem cells (BMSC), which can differentiate into adipocytes, chondrocytes, osteocytes, hepatocytes, cardiomyocytes and neurons; neural stem cells (NSC), which can differentiate into astrocytes, neurons, and oligodendrocytes; and peripheral blood stem cells. Multipotent stem cells have also been derived from epithelial and adipose tissues and umbilical cord blood (UCB).

ES cells, derived from the inner cell mass of preimplantation embryos, have been recognized as the most pluripotent stem cell population and are therefore the preferred cell for the methods of the invention. These cells are capable of unlimited proliferation ex vivo, while maintaining the capacity for differentiation into a wide variety of somatic and extra-embryonic tissues. ES cells can be male Q(Y) or female (XX); female ES cells are preferred.

Multipotent, adult stem cells can also be used in the methods of the disclosure. Preferred adult stem cells include hematopoietic stem cells (HSC), which can proliferate and differentiate throughout life to produce lymphoid and myeloid cell types; bone marrow-derived stem cells (BMSC), Which can differentiate into various cell types including adipocytes, chondrocytes, osteocytes, hepatocytes, cardiomyocytes and neurons; and neural stem cells (NSC), Which can differentiate into astrocytes, neurons, and oligodendrocytes. Multipotent stem cells derived from epithelial and adipose tissues and umbilical cord blood cells can also be used in the methods of the invention.

Stem cells can be derived from source, e.g., any mammal including, but not limited to, mouse, human, and primates. Following acquisition of stem cells, these cells can be used directly in the methods disclosed herein. For example, umbilical cord blood cells can be acquired in sufficient quantity to use directly for therapeutic purposes. Alternatively, stem cells can first be expanded in order to increase the number of available cells. See, for example, U.S. Pat. No. 6,338,942, content of which is incorporated herein by reference in its entirety. Exemplary mouse strains for stem cell preparation include 129, C57BL/6, and a hybrid strain (Brook et al., Proc. Natl. Acad. Sci. U.S.A. 94, 5709-5712 (1997), Baharvand et al., In Vitro Cell Dev. Biol. Anim. 40, 76-81 (2004), content of both of which is incorporated herein by reference). Methods for preparing mouse, human, or primate stem cells are known in the art and are described, for example, in Nagy et al., Manipulating the mouse embryo: A laboratory manual, 3rd ed., Cold Spring Harbor Laboratory Press (2002); Thomson et al., Science 282:1145-1147 (1998), Marshall et al., Methods Mol. Biol. 158: 1 1-18 (2001); Thomson et al., Trends Biotechnol. 18:5357 (2000); Jones et al., Semin. Reprod. Med. 18:219-223 (2000); Voss et al., Exp. Cell Res. 230:45-49 (1997); and Odorico et al., Stem Cells 19:193-204 (2001), content of all which is incorporated herein by reference in its entirety.

ES cells can be directly derived from the blastocyst or any other early stage of development, or can be a “cloned” stem cell line derived from somatic nuclear transfer and other similar procedures. General methods for culturing mouse, human, or primate ES cells from a blastocyst can be found in Appendix C of the NIH report on stem cells entitled Stem Cells: Scientific Progress and Future Research Directions (June 2001), content of which is incorporated herein by reference. For example, in the first step, the inner cell mass of a preimplantation blastocyst is removed from the trophectoderm that surrounds it. (For cultures of human ES cells, blastocysts are generated by in vitro fertilization and donated for research.) The small plastic culture dishes used to grow the cells contain growth medium supplemented with fetal calf serum, and are sometimes coated with a “feeder” layer of nondividing cells. The feeder cells are often mouse embryonic fibroblast (MEF) cells that have been chemically inactivated so they will not divide. Additional reagents, such as the cytokine leukemia inhibitory factor (LIF), can also be added to the culture medium for mouse ES cells. Second, after several days to a week, proliferating colonies of cells are removed and dispersed into new culture dishes, each of which may or may not contain an MEF feeder layer. If the cells are to be used to human therapeutic purposes, it is preferable that the MEF feeder layer is not included. Under these ex vivo conditions, the ES cells aggregate to form colonies. In the third major step required to generate ES cell lines, the individual, non-differentiating colonies are dissociated and replated into new dishes, a step called passage. This replating process establishes a “line” of ES cells. The line of cells is termed “clonal” if a single ES cell generates it. Limiting dilution methods can be used to generate a clonal ES cell line. Reagents needed for the culture of stem cells are commercially available, for example, from Invitrogen, Stem Cell Technologies, R&D Systems, and Sigma Aldrich, and are described, for example, in U.S. Patent Publication Nos. 2004/0235159 and 2005/0037492 and Appendix C of the NIH report, Stem Cells: Scientific Progress and Future Research Directions, supra. In some embodiments, the the stem cell is a human embryonic stem cell.

Combination Therapies

Wide variety compounds, e.g., therapeutic agents can have an additive or synergistic effect with the compound of Formula (I). Such compounds can additively or synergistically combine with the compound of Formula (I) in the methods disclosed herein, e.g., to differentiate stem cells and/or to treat a neurodegenerative disease or disorder and/or an inflammation or an inflammation-associated disease or disorder in a subject.

In some embodiments, a compound of Formula (I) can be used in combination with a second therapeutic agent useful in treating a neurodegenerative disease. In some embodiments, a compound of Formula (I) can be used in combination with a second therapeutic agent useful in treating a Parkinson's disease. In some embodiments, a compound of Formula (I) can be used in combination with a second therapeutic agent useful in treating an Alzheimer's disease. In some embodiments, a compound of formula (I) can be used in combination with a dopamine agonist. Without wishing to be bound by a theory, it is believed that the combination of a compound of Formula (I) with a dopamine agonist shows a synergistic effect on stimulating the transcriptional activity through the ligand binding domain of Nurr1 and enhancing the contrasting dual function of Nurr1. Exemplary analogs of dopamine include the ergolines and the aporphines such apomorphine, pergolide, bromocriptine and lisuride. Dopamine agonists are primarily used for the treatment of Parkinson's disease due to their neuroprotective effects on dopaminergic neurons.

Without wishing to be bound by a theory, a dopamine agonist can act via one of several pathways. For example, a dopamine agonist can activate or potentiate D1 dopamine receptors and/or Dj-like receptors such as D1 and D5 dopamine receptors and/or D2 dopamine receptors (e.g., D2, D2 short and D2 long receptors, D4, and D4 dopamine receptors) and/or D3 dopamine receptors and/or D4 dopamine receptors. A dopamine agonist can act by inhibiting one or more enzyme involved in biosynthesis and/or transformation and/or breakdown of dopamine.

Exemplary dopamine agonists include, but are not limited to, L-3,4-dihydroxyphenylalanine (L-Dopa); (−)-7-{[2-(4-Phenylpiperazin-1-yl)ethyl]propylamino}-5,6,7,8-tetrahydronaphthalen-2-ol; (+)-4-propyl-9-hydroxynaphthoxazine ((+)PHNO); (E)-1-aryl-3-(4-pyridinepiperazin-1-yl)propanone oximes; (R)-3-(4-Propylmorpholin-2-yl)phenol (PF-219,061); (R,R)-S32504; 2-(N-phenylethyl-N-propylamino)-5-hydroxytetralin; 2-bromo-a-ergocriptine (bromocriptine); 5,6,7,8-Tetrahydro-6-(2-propen-1-yl)-4H-thiazolo[4,5-d]azepin-2-amine (BHT-920); 5-HT uptake inhibitor; 5-HT-1A agonists (such as roxindole); 6-Br-APB; 6-methyl-8-a-(N-acyl)amino-9-ergoline; 6-methyl-8-a-(N-phenyl-acety)amino-9-ergoline; 6-methyl-8β-carbobenzyloxy-aminoethyl-10-a-ergoline; 7,8-Dihydroxy-5-phenyl-octahydrobenzo[h]isoquinoline; 8-acylaminoergoline; 9,10-dihydroergocomine; a2-adrenergic antagonist (such as terguride); A-412,997; A-68,930; A-77,636; A-86,929; ABT-670; ABT-724; AF-14; alaptide; amisulpride; any D-2-halo-6-alkyl-8-substituted ergoline; Aplindore; Apomorphine; Aripiprazole (Abilify in USA); benzazepine analogs; BP-897; Bromocriptine; bromocriptine mesylate; Cabergoline; cis-8-Hydroxy-3-(n-propyl)-1,2,3a,4,5,9b-hexahydro-1H- and trans-N-{4-[4-(2,3-Dichlorophenyl)-1-piperazinyl]cyclohexyl}-3-methoxybenzamide; clozapine; COMT inhibitors (such as CGP-28014, entacapone and tolcapone); CP-226,269; CP-96,345; CY-208,243; D-2-bromo-6-methyl-8-cyanomethylergoline; Dihydrexidine; dihydro-alpha-ergocriptine; dihydro-alpha-ergotoxine; dihydroergocriptine; dihydroergocryptine; dihydroergotoxine (hydergine); Dinapsoline; Dinoxyline; domperidone; Dopamine; dopamine D1 receptor agonists; dopamine D2 receptor agonists; dopamine D3 receptor agonists; dopamine D4 receptor agonists; dopamine D5 receptor agonists; dopamine uptake inhibitors (such as GBR-12909, GBR-13069, GYKI-52895, and NS-2141); doprexin; Doxanthrine; ER-230; erfotoxine; Ergocornine; ergoline derivatives; ergot alkaloid derivatives;

eticlopride; etisulergine; FAUC 299; FAUC 316; Fenoldopam; Flibanserin; haloperidol; iloperidone; levodopa; Lisuride; lisuride; LSD; LU111995; mazapertine; Methylphenidate; monoamine oxidase-B inhibitors (such as selegiline, N-(2butyl)-N-methylpropargylamine, N-methyl-N-(2-pentyl) propargylamine, AGN-1133, ergot derivatives, lazabemide, LU-53439, MD-280040 and mofegiline); N-0434; Naxagolide; olanzapine; opiate receptor agonists (such as NIH-10494); PD-118,440; PD-168,077; Pergolide (such as A-68939, A-77636, dihydrexine, and SKF-38393); PIP3EA; piribedil; Piribedil; Pramipexole; Quinagolide; Quinelorane; Quinpirole; racemic trans-10,11-dihydroxy 5,6,6a, 7,8,12b-hexahydro and related benzazepine analogs; raclopride; remoxipride; risperidone; Ro10-5824; Ropinirole; Rotigotine; Salvinorin A; SDZ-HDC-912; sertindole; SKF-38,393; SKF-75,670; SKF-81,297; SKF-82,526 (fenoldopam); SKF-82,598; SKF-82,957; SKF-82,958; SKF-38,393; SKF-77,434; SKF-81,297; SKF-82,958; SKF-89,145; SKF-89,626; spiperone; spiroperidol; sulpride; sumanirole; Talipexole; Terguride; tropapride; WAY-100635; YM 09151-2; zetidoline; β-adrenergic receptor agonists; cabergoline; bromocriptine; pergolide; talipexole; ropinirole; pramipexole; and analogs, derivatives, enantiomers, metabolites, prodrugs, and pharmaceutically acceptable salts thereof.

In some embodiments, a compound of formula (I) can be used in combination with a beta-3 adrenergic receptor agonist. Exemplary beta-3 adrenergic receptor agonists include, but are not limited to, DPDMS; dopexamine; AJ-9677; AZ-40140; BMS187413; BMS-194449; BMS-210285; BRL-26830A; BRL-28410; BRL-35135; BRL-37344; CGP 12177; CL-316243; CP-114271; CP-331648; CP-331679; D-7114; FR-149175; GW-2696; GW-427353; ICI-198157; L-750355; L-796568; LY-377604; N-5984; SB-226552; SR-58611A; SR-59062A; SWR0342SA; ZD-2079; and analogs, derivatives, enantiomers, metabolites, prodrugs, and pharmaceutically acceptable salts thereof.

In some embodiments, the dopamine agonist inhibits the dopamine beta-hydroxylase. Dopamine beta-hydroxylase converts dopamine to norepinephrine. Thus, by inhibiting dopamine beta-hydroxylase, intracellular dopamine is increased while norepinephrine is decreased.

In some embodiments, a compound of Formula (I) can be used in combination with an inhibitor of a dopamine beta-hydroxylase. Exemplary inhibitors of DBH include, but are not limited to fusaric acid; 1,1′,1″,1′″-[disulfanediylbis-(carbonothioylnitrilo)]tetraethane (disulflram); 2-Hydroxy-2,4,6-cycloheptatrien-1-one (tropolone, also referred to as 2-Hydroxytropone or Purpurocatechol); 5-(aminomethyl)-1-[(2 S)-5,7-difluoro-1,2,3,4-tetrahydronaphthalen-2-yl]-1,3-dihydro-2 H -imidazole-2-thione (Nepicastat, INN, or SYN117)); 1-(4-hydroxybenzyl)imidazole-2-thiol; FLA-63; diethyidithiocarbamate; betachlorophenethylamine; 4-hydroxybenzyl cyanide; 2-halo-3(p-hydroxyphenyl)-1-propene; 1-phenyl-1-propyne; 2-phenylallylamine; 2-(2-thienyl)allylamine; 2-thiophene-2(2-thienyl)allylamine; 3-phenylpropargylamine; 1-phenyl-1 (aminoethyl)ethane; N-(trifluoroacetyl)phenyl(aminoethyl) ethane; 5-picolinic acid substituted with an alkyl group containing up to 6 carbon atoms; 5-picolinic acid substituted with a halo alkyl group containing up to 6 carbon atoms; and analogs, derivatives, enantiomers, metabolites, prodrugs, and pharmaceutically acceptable salts thereof. Other inhibitors of dopamine beta-hydroxylase include, but are not limited to U.S. Pat. Nos. 4,487,761; 4,634,711; 4,719,223; 4,743,613; 4,749,717; 4,761,415; 4,762,850; 4,798,843; 4,810,800; 4,835,154; 4,839,371; 4,859,779; 4,876,266; 4,882,348; 4,906,668; 4,935,438; 4,963,568; 4,992,459; 5,100,912; 5,189,052; 5,597,832; 6,407,137; 6,559,186; 7,125,904; 7,576,081, content of all of which is herein incorporated by reference in their entirety.

Any of the following compounds can be co-administered with a compound of Formula (I). Cabergoline is a long-acting ergot derivative agonist with a high affinity for D2 receptors. Bromocriptine is an ergot alkaloid dopamine receptor agonist. It is a strong D2 receptor agonist and a weak D1 receptor antagonist. It stimulates both pre-and post-synaptic receptors. Pergolide is a semisynthetic, clavine ergot alkaloid dopamine agonist. In contrast to bromocriptine, it is a strong D2 receptor agonist and a weak Dl receptor agonist. Ropinirole is a potent, non-ergoline dopamine agonist. Pramipexole is a synthetic amino-benzothiazol derivative and a non-ergot D2/D3 agonist. Quinagolide is another, non-ergot, non-ergoline, benzoquinoline dopaminergic agonist that blocks prolactin release. In some embodiments, a compound of Formula (I) can be administered to the subject in combination with a second therapeutic agent useful in treating a Parkinson's disease. Examples such second therapeutic agents include carbidopa-levodopa, MAO B inhibitors (selegiline, rasagiline, or safinamide), catechol O-methyltransferase (COMT) inhibitors (e.g., entacapone), anticholinergics (benztropine or trihexyphenidyl), and amantadine. The administration of the compound of Formula (I) can also be combined with a surgical procedure, such as deep brain stimulation.

For treating inflammation or inflammation-associated disorders, a compound of Formula (I) can be co-administered with an agent known in the art for treatment of inflammation or inflammation-associated disorders or infections. Exemplary anti-inflammatory agents include non-steroidal anti-inflammatory drugs (NSAIDs—such as aspirin, ibuprofen, or naproxen, corticosteroids (such as prednisone), an antimalarial medication (such as hydrochloroquine), methotrexate, sulfasalazine, leflunomide, anti-TNF medications, cyclophosphamide, mycophenolate, dexamethasone, rosiglitazone, prednisolone, corticosterone, budesonide, estrogen, estradiol, sulfasalazine, fenofibrate, pravastatin, simvastatin, pioglitazone, acetylsalicylic acid, mycophenolic acid, mesalamine, and analogs, derivatives, prodrugs, and pharmaceutically acceptable salts thereof. In some embodiments, the compound of Formula (I) can be co-administered with forskoline, amodiaquine (AQ), chloroquine (CQ), or glafenine. For treating infectious diseases, the compound of Formula (I) can be co-administered with a second therapeutic agent useful in treating the infectious disease or disorder. For example, the compound of Formula (I) can be co-administered with atovaquone, proguanil, quinine, doxycycline, mefloquine, or primaquine, or any pharmaceutically acceptable salt or any combination thereof.

Without limitations, the compound of Formula (I) and the second compound can be administered in the same formulation or in separate formulations. When administered in separate formulations, the compound of Formula (I) and the second compound can be administered within any time of each other. For example, the compounds can be administered within 24 hours, 12 hours, 6 hours, 5 hours, 4 hours, 3 hours, 2 hours, 1 hours, 45 minutes, 30 minute, 25 minutes, 20 minutes, 15 minutes, 10 minutes, 5 minutes or less of each other. When administered in separate formulations, either compound can be administered first.

Additionally, co-administration does not require the two compounds to be administered by the same route. As such, each can be administered independently or a common dosage form. Further, the two compounds can be administered in any ratio to each other by weight or moles. For example, the two compounds can be administered in a ratio of from about 50:1, 40:1, 30:1, 25:1, 20:1, 15:1, 10:1, 5:1, 3:1, 2:1, 1:1.75, 1.5:1, or 1.25:1 to 1:1.25, 1:1.5, 1.75, 1:2, 1:3, 1:4, 1:5, 1:10, 1:15, 1:20, 1:20, 1:30, 1:40, or 1:50. The ratio can be based on the effective amount of either compound. In some embodiments, a compound of Formula (I) can be co-administered with forskolin or colfosin. In some embodiments, amodiaquine or chloroquine can be co-administered with forskolin or colfosin. In some embodiments, a compound of Formula (I) is co-administered with amodiaquine or chloroquine, can be further co-administered with a dopamine agonist.

Pharmaceutical Compositions and Formulations

The present application also provides pharmaceutical compositions comprising an effective amount of a compound of Formula (I), or a pharmaceutically acceptable salt thereof; and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

Dosages and Regimens

In the pharmaceutical compositions of the present application, a compound of Formula (I) is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of the compound of Formula (I) can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound of Formula (I) is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

Kits

The present invention also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent as described herein.

Definitions

As used herein, the term “about” means “approximately” (e.g., plus or minus approximately 10% of the indicated value).

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the cell with a compound of the invention includes the administration of a compound of the present invention to an individual or patient, such as a human, having the cell, as well as, for example, introducing a compound of the invention into a sample containing a cellular or preparation.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician. As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

As used herein, the term “stem cell” is meant any cell with the potential to self-renew and, under appropriate conditions, differentiate into a dedicated progenitor cell or a specified cell or tissue. Stem cells can be pluripotent or multipotent. Stem cells include, but are not limited to embryonic stem cells, embryonic germ cells, adult stem cells, and umbilical cord blood cells.

As used herein, the term “inflammation” refers to any cellular processes that lead to the activation of caspase-1, or caspase-5, the production of cytokines IL-I, IL-6, IL-8, TNF-alpha, iNOS, and/or the related downstream cellular events resulting from the actions of the cytokines thus produced, for example, fever, fluid accumulation, swelling, abscess formation, and cell death. As used herein, the term “inflammation” refers to both acute responses (i.e., responses in which the inflammatory processes are active) and chronic responses (i.e., responses marked by slow progression and formation of new connective tissue)., Acute and chronic inflammation may be distinguished by the cell types involved. Acute inflammation often involves polymorphonuclear neutrophils; whereas chronic inflammation is normally characterized by a lymphohistiocytic and/or granulomatous response.

As used herein, the term “pathogen infection” refers to infection with a pathogen. As used herein the term “pathogen” refers to an organism, including a microorganism, which causes disease in another organism (e.g., animals and plants) by directly infecting the other organism, or by producing agents that causes disease in another organism (e.g., bacteria that produce pathogenic toxins and the like). As used herein, pathogens include, but are not limited to bacteria, protozoa, fungi, nematodes, viroids and viruses, or any combination thereof; wherein each pathogen is capable, either by itself or in concert with another pathogen, of eliciting disease in vertebrates including but not limited to mammals, and including but not limited to humans. As used herein, the term “pathogen” also encompasses microorganisms which may not ordinarily be pathogenic in a non-immunocompromised host. Specific nonlimiting examples of viral pathogens include Herpes simplex virus HSV1, HSV2, Epstein Barr virus (EBV), cytomegalovirus (CMV), human Herpes virus HHV6, HHV7, HHV8, Varicella zoster virus (VZV), hepatitis C, hepatitis B, HIV, adenovirus, Eastern Equine Encephalitis Virus (EEEV), West Nile virus (WNE), JC virus (JCV) and BK virus (BKV).

As used herein, the term “microorganism” includes prokaryotic and eukaryotic microbial species from the Domains of Archaea, Bacteria and Eucarya, the latter including yeast and filamentous fungi, protozoa, algae, or higher Protista. The terms “microbial cells” and “microbes” are used interchangeably with the term microorganism.

As used herein, the term “bacteria” refers to a domain of prokaryotic organisms. Bacteria include at least 11 distinct groups as follows: (1) Gram-positive (gram+) bacteria, of which there are two major subdivisions: (i) high G+C group (Actinomycetes, Mycobacteria, Micrococcus, others) (ii) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) cyanobacteria, e.g., oxygenic phototrophs; (4) Spirochetes and related species; (5) Planctomyces; (6) Bacteroides, Flavobacteria; (7) chlamydia; (8) Green sulfur bacteria; (9) Green non-sulfur bacteria (also anaerobic phototrophs); (10) Radioresistant micrococci and relatives; (11) thermotoga and thermosipho thermophiles.

As used herein, the term “Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

As used herein, the term “Gram-positive bacteria” include cocci, nonsporulating rods, and sporulating rods. The genera of Grampositive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

As used herein, the term “specific defense system” is intended to refer to that component of the immune system that reacts to the presence of specific antigens. Inflammation is said to result from a response of the specific defense system if the inflammation is caused by, mediated by, or associated with a reaction of the specific defense system. Examples of inflammation resulting from a response of the specific defense system include the response to antigens such as rubella virus, autoimmune diseases such as lupus erythematosus, rheumatoid arthritis, Reynaud's syndrome, multiple sclerosis etc., delayed type hypersensitivity response mediated by T-cells, etc. Chronic inflammatory diseases and the rejection of transplanted tissue and organs are further examples of inflammatory reactions of the specific defense system.

As used herein, a reaction of the “non-specific defense system” is intended to refer to a reaction mediated by leukocytes incapable of immunological memory. Such cells include granulocytes and macrophages. As used herein, inflammation is said to result from a response of the nonspecific defense system, if the inflammation is caused by, mediated by, or associated with a reaction of the non-specific defense system. Examples of inflammation which result, at least in part, from a reaction of the non-specific defense system include inflammation associated with conditions such as: adult respiratory distress syndrome (ARDS) or multiple organ injury syndromes secondary to septicemia or trauma; reperfusion injury of myocardial or other tissues; acute glomerulonephritis; reactive arthritis; dermatoses with acute inflammatory components; acute purulent meningitis or other central nervous system inflammatory disorders; thermal injury; hemodialysis; leukophoresis; ulcerative colitis; Crohn's disease; necrotizing enterocolitis; granulocyte transfusion associated syndromes; and cytokine-induced toxicity. The term immune-mediated refers to a process that is either autoimmune or inflammatory in nature.

The term “synergistic” as used herein is defined to mean a combination of components wherein the activity of the combination is greater than the additive of the individual activities of each component of the combination. In some embodiments, the activity of the combination is at least 5%, at least 10%, at least 15%, at least 20%, at least 25%, at least 30%, at least 40%, at least 50%, at least 60%, at least 70%, at least 80%, at least 90%, at least 1-fold, at least 2-fold, at least 3-fold, at least 4-fold, at least 5-fold, at least 10-fold, at least 50-fold, at least 100-fold or greater than the additive of the individual activities of each component of the combination.

As used herein, the terms “Nurr1 nucleic acid” and “Nurr1 gene” are used interchangeably herein and refer to a nucleic acid that encodes all or a portion of a Nurr1 polypeptide, or is substantially identical to all or a portion of the nucleic acid sequence of Genbank Accession No. ABOI 7586 (Ichinose et al., Gene 230:233-239, 1999), or analog thereof.

As used herein, the term “Nurr1 polypeptide” is meant a polypeptide substantially identical to all or a portion of the polypeptide sequence of Genbank Accession No. BAA75666, or analog thereof, and having Nurr1 biological activity.

### EXAMPLES

### Example 1

### SPV-94 is a Functional Nurr1 Activator

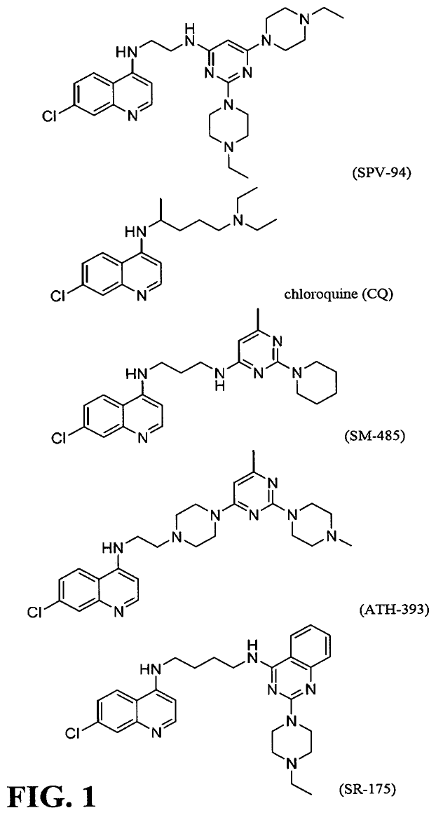

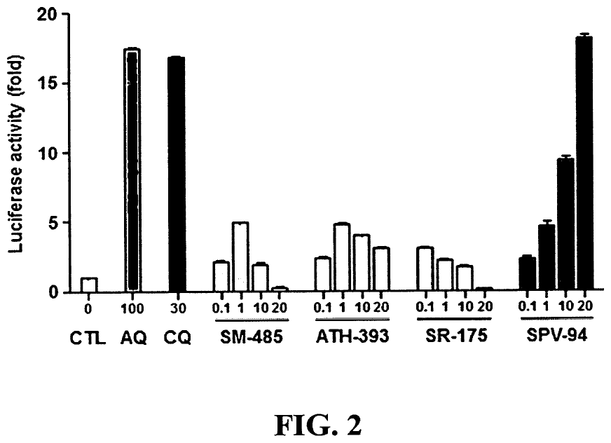

In vitro assays, including luciferase activity assay using p4xNL3-Luc reporter construct along with Nurr1 full-length or ligand binding domain (LBD) expression plasmid (See, Kim et al., PNAS, 2015, 112, 8756-8761), cytotoxicity assay measuring lactate dehydrogenase (LDH) release, and immune suppression assay by real-time PCR for inflammatory gene expressions, showed that SPV-94 has superior Nurr1activating properties when compared to control, AQ, CQ, and compounds SM-485, ATH-393 and SR-175 (previously disclosed in WO 2013/134047) (see FIGS. 1 and 2).

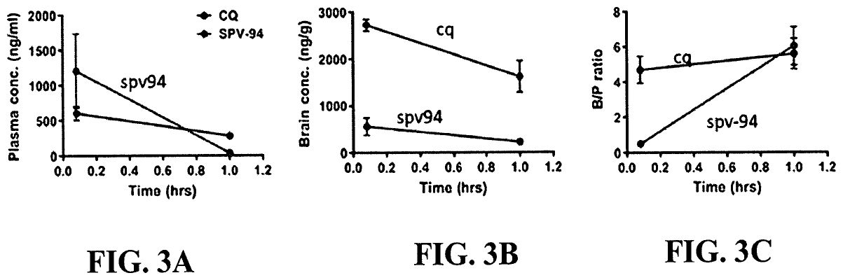

Brain permeability and characterization of SPV-94 was evaluated by LC-MS/MS (liquid chromatography-tandem mass spectrometry) analysis of blood plasma and brain homogenates 0.8 and 1 hr after intravenous (i.v.) injection in rats (5 mg/kg). SPV-94 penetrated into the brain slower than CQ and consistently maintained its level (See FIG. 3). cLogP for SPV-94 is 1.952, and solubility is 504.88 μM at pH 7.4.

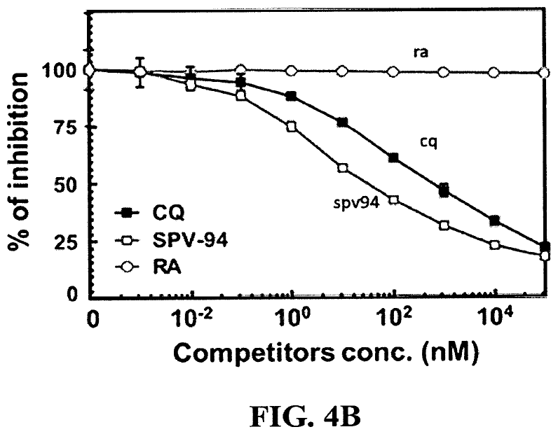

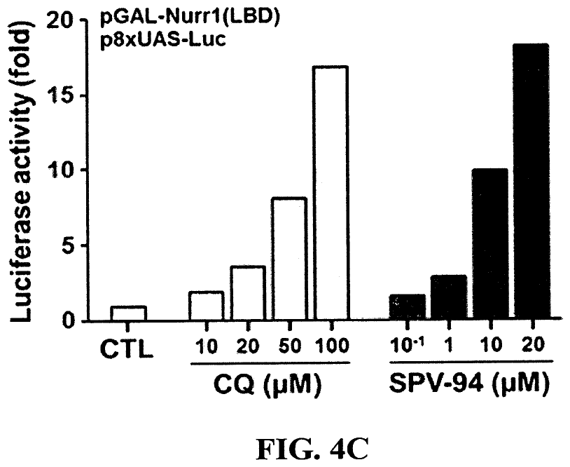

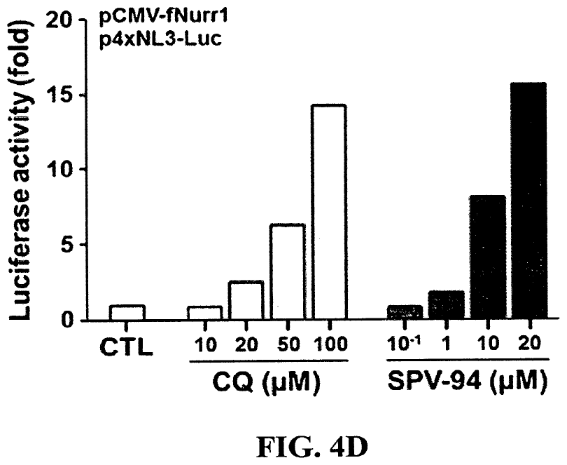

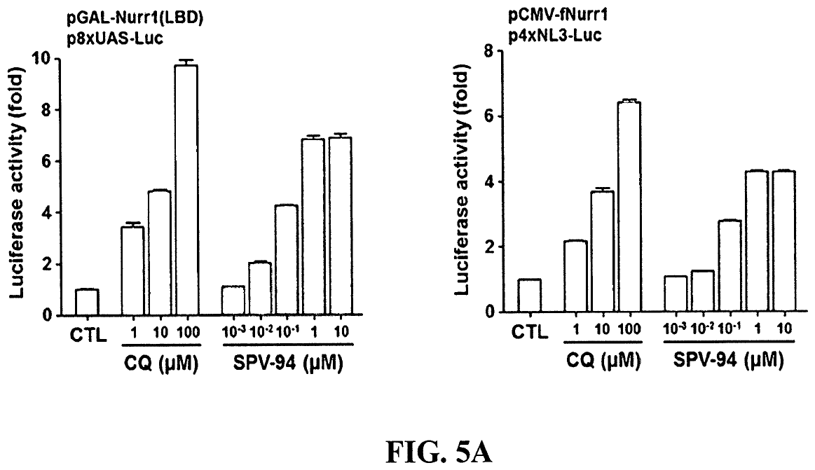

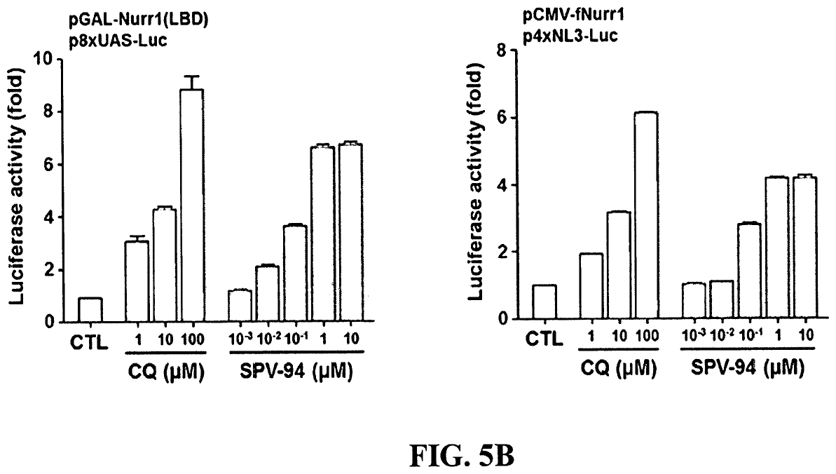

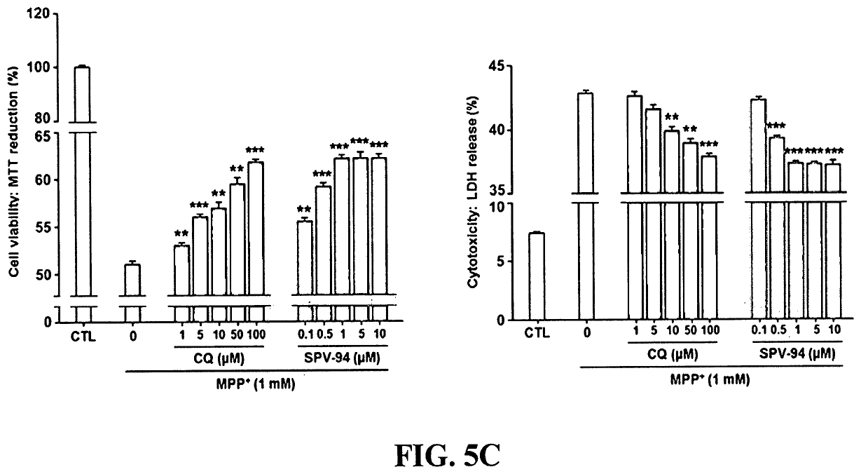

Radioligand binding assay using [3H]-CQ revealed that SPV-94 and CQ can compete with [3H]-CQ for binding to Nurr1-LBD with Kᵢ values of 11.09 and 28.42 nM, respectively (FIG. 4B). As shown in FIG. 4B, SPV-94 exhibited 20 times higher affinity to Nurr1-LBD based on their IC₅₀s were 1 μM and 50 nM for CQ and SPV-94, respectively. SPV-94 increased transcriptional activities of both Nurr1-LBD and full-length Nurr1 in a dose-dependent manner, with a higher efficiency than CQ at a 5 times lower concentration in SK-N-BE(2) human neuroblastoma cell line (EC₅₀˜10 μM; FIGS. 4C and 4D). SPV-94 exhibited even ˜1,000 times lower EC₅₀ than CQ in the luciferase activity assay using murine-derived dopaminergic cell lines, MN9D and N27-A (EC₅₀ ˜50 nM; FIGS. 5A and 5B).

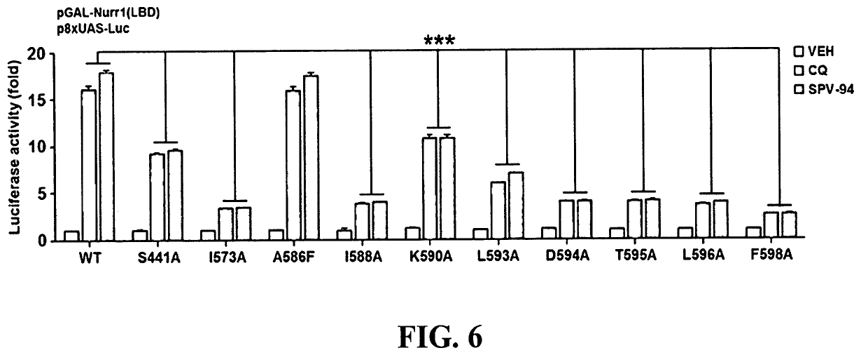

To address whether SPV-94 functions through Nurr1, Nurr1 transactivation was assessed using point-mutant form of Nurr1 LBD at the perturbed residues based on the preliminary NMR titration of ¹⁵N-labeled Nurr1-LBD in the presence of CQ. Significant reduction of transcriptional activity with mutations at 1573, 1588, L593, D594, T595, L596 and F598 revealed that these sites are critical for interaction between Nurr1 and CQ/SPV-94 for its activation, demonstrating CQ and SPV-94 actions by direct binding to Nurr1-LBD (FIG. 6).

### Example 2

### Neuroprotective Effects of SPV-94 Via Nurr1

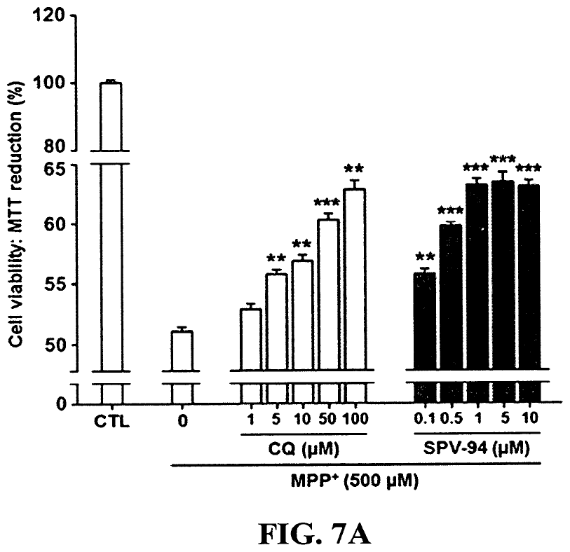

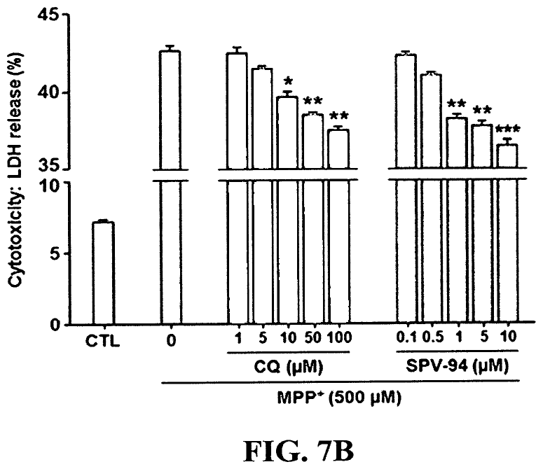

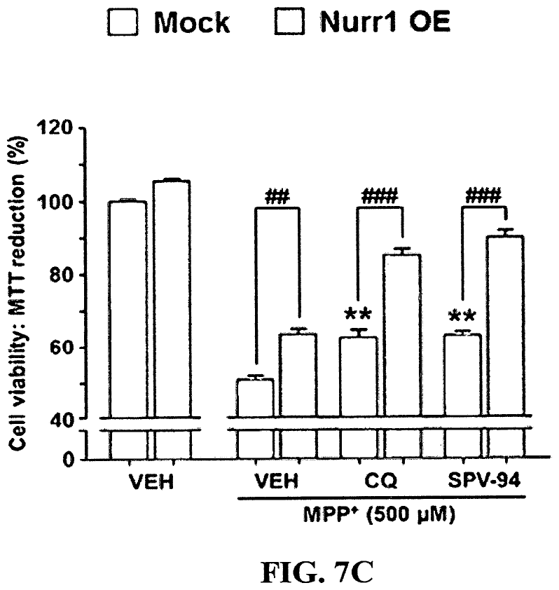

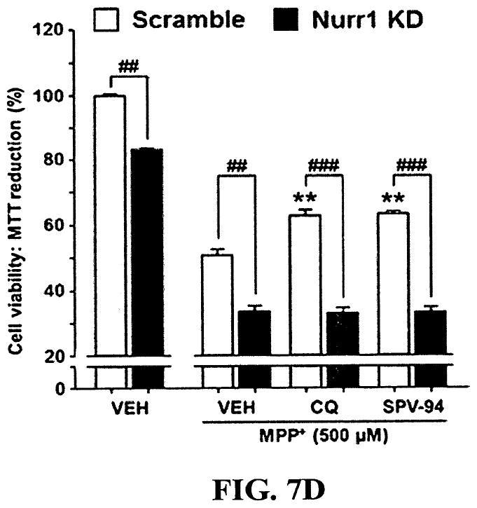

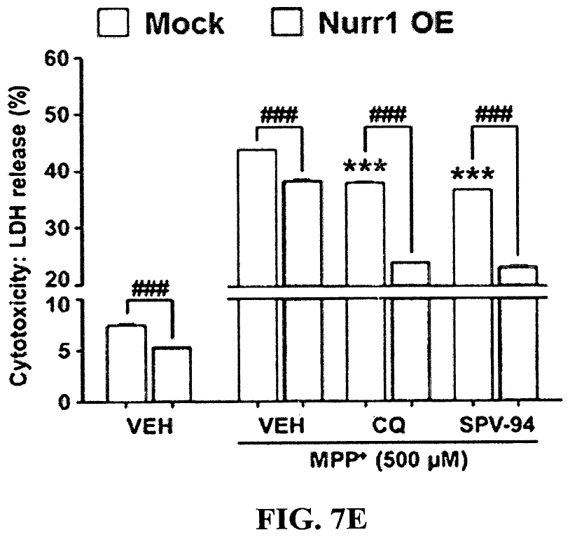

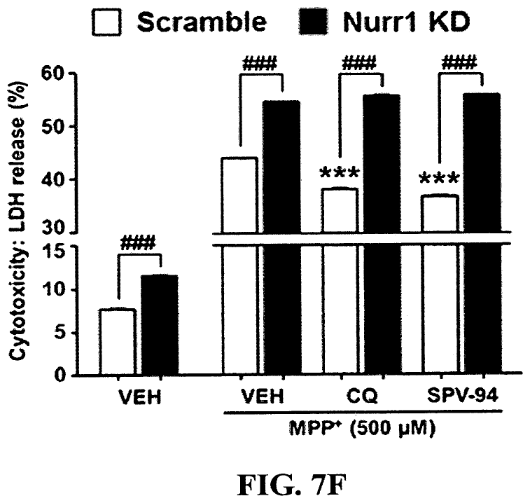

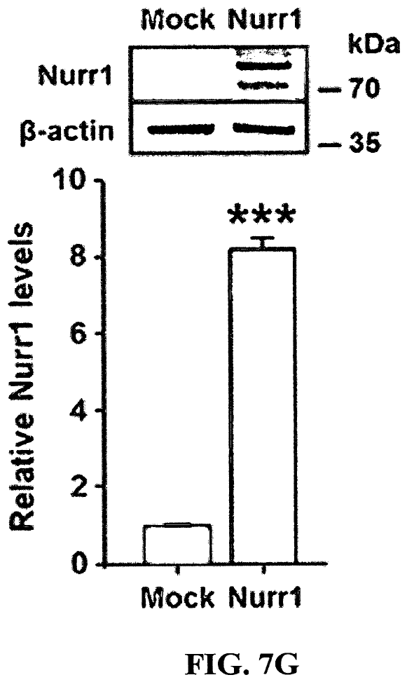

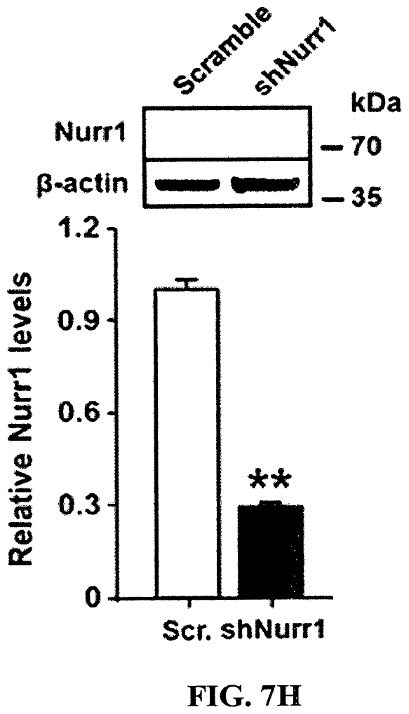

To determine whether Nurr1 activation by CQ and SPV-94 exerts neuroprotective effects, CQ and SPV-94 were tested in MN9D and N27-A cells in which PD-like toxic condition was induced by mitochondrial complex I inhibitor 1-methyl-4-phyenylpyridinium (MPP+). CQ and SPV-94 significantly decreased MPP⁺-induced cytotoxicity both in MN9D (FIG. 7A and 7B) and N27-A dopaminergic cell lines (FIG. 5C) in a dose-dependent manner. Remarkably, SPV-94 showed its maximal efficiency at 10 times lower concentration than CQ.

Next it was tested whether this neuroprotection is Nurr1-dependent using Nurr1 overexpression (OE) or knockdown (KID) in MN9D cells. Nurr1 OE potentiated the neuroprotective effects of CQ and SPV-94 against MPP+toxicity (FIG. 7C and 7E), meanwhile Nurr1 KD abrogated it (FIG. 7D and 7F).

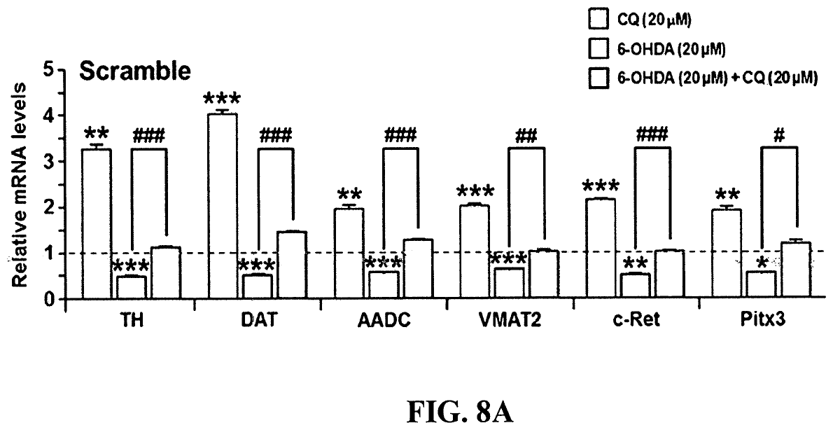

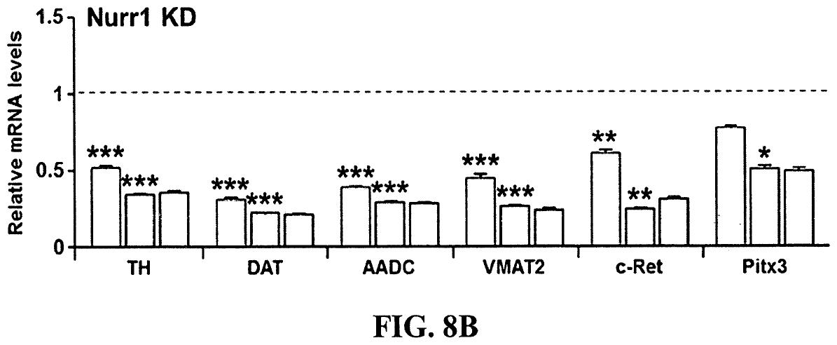

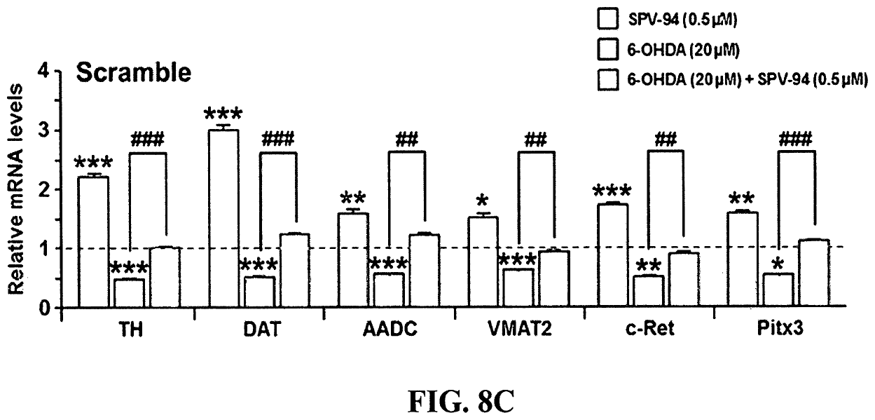

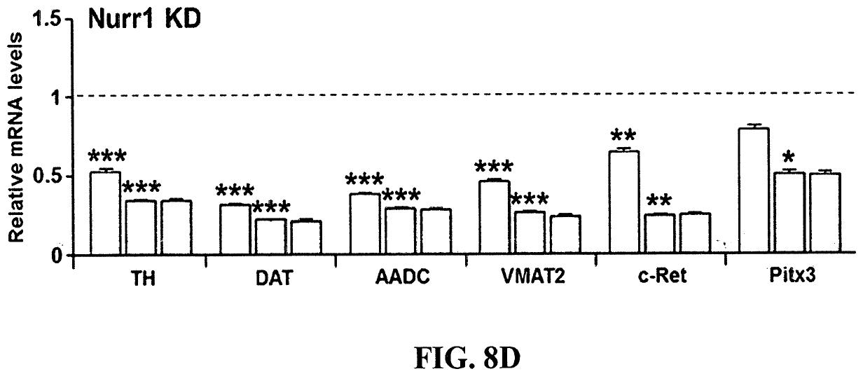

Regulatory effects of CQ and SPV-94 on dopaminergic (DAergic) gene transcriptions were further examined in the absence or presence of PD-like toxicity. In mouse embryonic ventral mesencephalic (mVM) primary neurons derived from embryonic day 12.5 (E12.5), CQ (20 μM) and SPV-94 (0.5 μM) significantly increased expressions of DA metabolism and maintenance related genes including tyrosine hydroxylase (TH), dopamine transporter (DAT), aromatic amino acid decarboxylase (AADC), vesicular monoamine transporter 2 (VMAT2), c-Ret receptor tyrosine kinase and paired like homeodomain 3 (Pitx3), which are known as Nurr1 target genes in the midbrain DAergic neurons (See Jacobs et al., Development 2009, 136:2363-2373) (FIG. 8A and 8C). Even more, CQ and SPV-94 retained expressions of these genes against 6-OHDA treatment. However, the neuroprotective effects of CQ and SPV-94 were disappeared when Nurr1 was knocked down (FIG. 8B and 8D), suggesting that DAergic genes regulation by CQ and SPV-94 is Nurr1-dependent.

### Example 3

### Immune Suppressive Effects of SPV-94 (Via Nurr1)

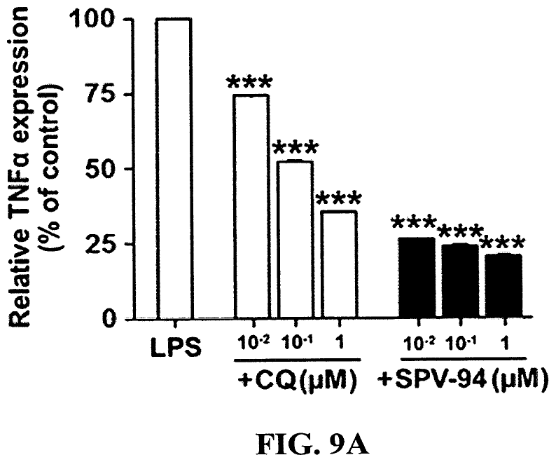

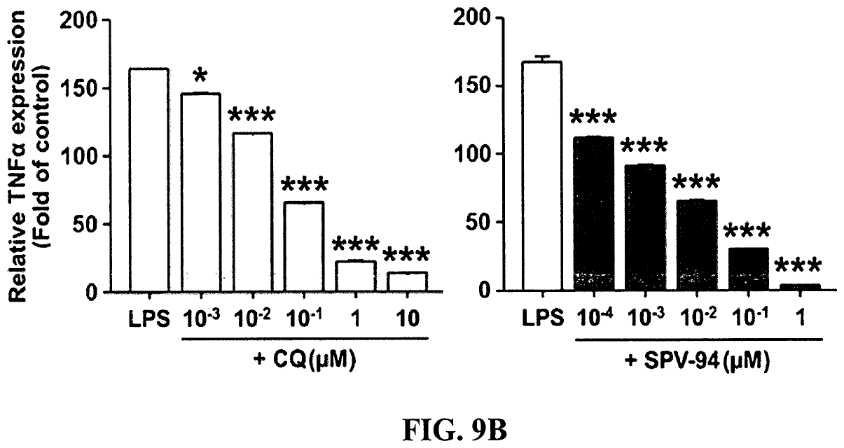

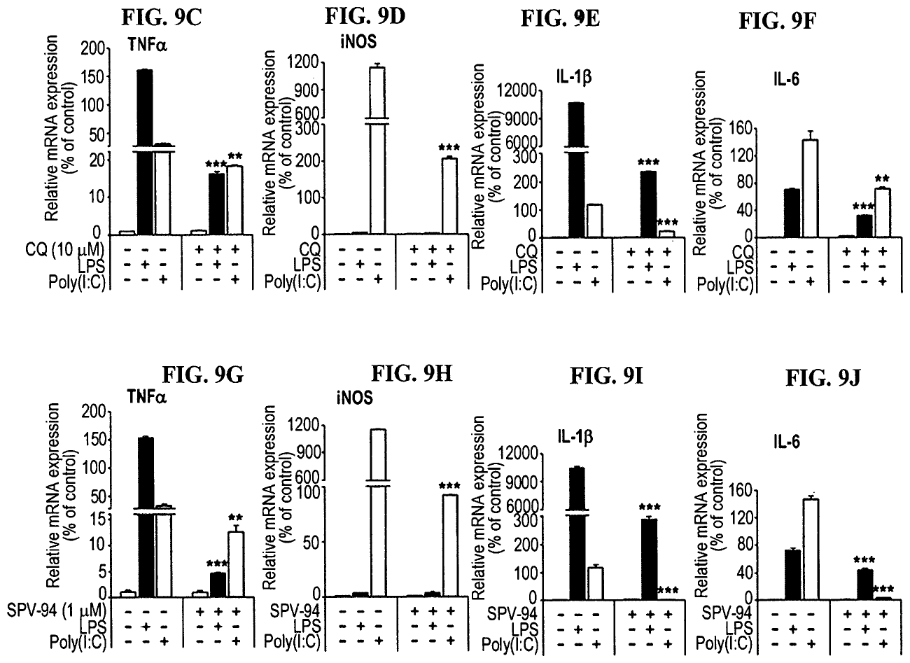

Besides the role as an activator of DAergic genes, Nurr1 is known as a repressor of inflammatory genes in astrocytes and microglia (See Saijo et al., Cell 2009, 137, 47-59). To investigate whether the immune suppressive function of Nurr1 is modulated by CQ and SPV-94, inflammation was induced in mouse microglia-derived BV2 cell line and mouse bone marrow-derived primary macrophages (mBMMs) by treating bacterial lipopolysaccharide (LPS) or a synthetic double-stranded RNA (dsRNA) polyinosinic-polycytidylic acid (poly(I:C)) which activate inflammation via toll-like receptor (TLR) 4 or 3, respectively. First, tumor-necrosis factor-α (TNFα) was dramatically induced by LPS treatment in BV2 and mBMMs. But notably, CQ and SPV-94 robustly suppressed TNFa expression dose-dependently, down by 35.53 and 20.67% at 1 μM respectively compared to LPS treated group in BV2 cells (FIG. 9A). This immune suppressive effects by CQ and SPV-94 were significantly dose-dependent in mBMMs as well, showing that SPV-94 has ˜10 times lower EC₅₀ than CQ (FIG. 9B).

The effects of CQ and SPV-94 on suppression of other pro-inflammatory gene expressions were further analyzed including inducible nitric oxide synthase (iNOS), interleukin 1-beta (IL-1β and interleukin-6 (IL-6) against LPS or poly(I:C) stimulation in mBMMs. LPS and poly(I:C) differently induced pro-inflammatory gene expressions, but all four genes were significantly downregulated in the presence of CQ (10 μM) or SPV-94 (1 μM) (FIGS. 9C-9J).

### Example 4

### Preserved Autophagy and Stabilized Nurr1 Expression by SPV-94

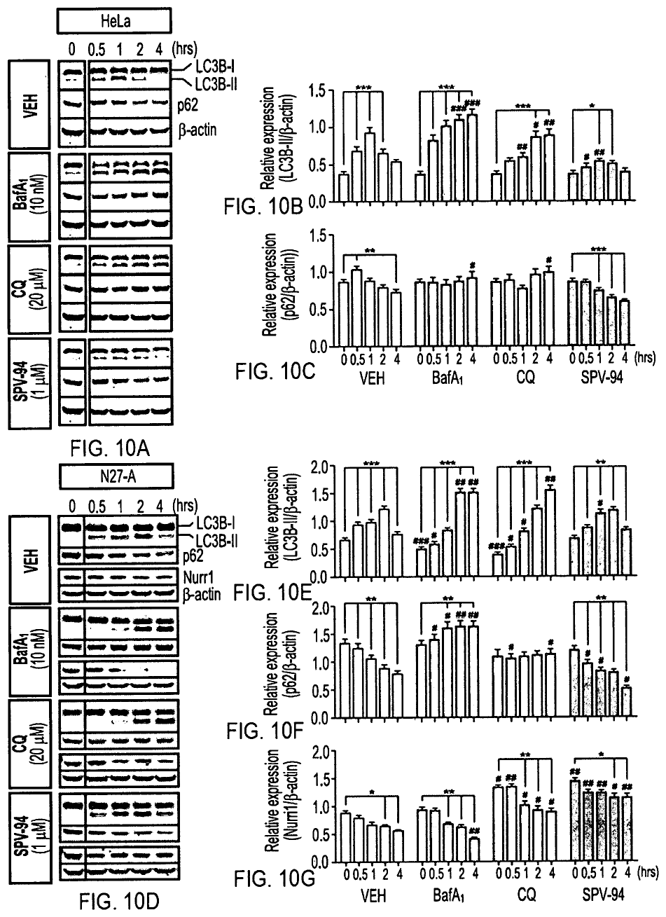

Since CQ is known as an autophagy inhibitor as disrupting fusion of mature autophagosome and lysosome (late-stage of autophagy process) (See Kimura et al., Cancer Res 2012, 73, 3-7; Al-Bari, J Antimicrob Chemother 2015, 70, 1608-1621; Yoshida, J Hematol Oncol 2017, 10, 67) and dysregulated autophagy is implicated in PD pathology (See Menzies et al., Neuron 2017, 93:1015-1034; Scrivo et al., Lancet Neurol 2018, 17:802-815), in this example it was determined whether SPV-94 also affects autophagy process or not. To validate and compare the autophagy regulation by CQ and SPV-94, first autophagy was induced by starvation in HeLa cells which is widely used for autophagy monitoring (See Tanida et al., Autophagy 2005, 1, 84-91; Klionsky et al., Autophagy 2012, 8, 445-544; Nguyen et al., J Cell Biol 2016, 215, 857-874). The expression changes of two autophagic markers LC3B and p62 indicated that CQ could initiate but failed to terminate autophagy process similarly to bafilomycin A₁ (BafA₁), which is another well-known autophagy blocker that interrupts at the late-state of autophagy process (FIGS. 10A-10C). Interestingly, SPV-94 successfully finalize autophagy showing decreased p62 expression (FIG. 10C).

Next, autophagy regulation was assessed in a dopaminergic cell line N27-A in the absence or presence of MPP⁺ (1 mM). Similar as observed in HeLa cells, autophagy was successfully terminated by SPV-94 treatment but not by BafA₁ or CQ treatment (FIGS. 10D-10F), suggesting that SPV-94 does not disrupt autophagy process. Furthermore, it was observed that MPP disrupted autophagy showing accumulated LC3B-II and remained p62 at the late-stage, which corresponds to the previous studies (See Lim et al., Autophagy 2011, 7, 51-60; Hung et al., PLoS ONE 2014, 9, e91074; Park et al., J Biol Chem 2016, 291, 3531-3540). Importantly, SPV-94 treatment seemed to protect N27-A cells from MPP+-induced autophagy dysregulation leading to successful p62 degradation (FIG. 10F).

When it was additionally determined that Nurr1 expression changes throughout autophagy process in N27-A cells, it showed gradual decrease in line with the autophagic degradation. Remarkably, CQ and SPV-94 significantly increased basal expression level of Nurr1, and due to its higher initial expression in CQ or SPV-94 treated condition, Nurr1 level remained significantly higher than in VEH group at the late-stage of autophagy process (FIG. 10G).

Referring to FIGS. 10D-10G, N27-A cells were treated with vehicle or MPP+ (1 mM) for 24 hrs and then changed with fresh growth medium with or without MPP+ 1 hr before starvation. BafA1 (10 nM), CQ (20 μM), or SPV-94 (1 μM) were treated for 4 hrs before autophagy induction. To induce autophagy, N27-A cells were incubated in EBSS for 0-4 hrs, in the absence or presence of MPP+. Samples were analyzed by Western blot using autophagic flux markers LC3B and p62 (D) and its expression levels were quantified (10E and 10F). Autophagy was superfluously induced but not successfully terminated by MPP+ treatment. (10G) Nurr1 expression level changes were also analyzed and quantified. Interestingly, CQ and SPV-94 treatments significantly increased Nurr1 expression compared to VEH. Notably, SPV-94 maintained Nurr1 expression against MPP+. *p<0.05, **p<0.01, ***p<0.001; #p<0.05, ##p<0.01, ###p<0.001 compared to VEH, one-way ANOVA, Tukey's multiple comparisons.

### Example 5

### Rescue of Behavioral and Pathophysiological Deficits by SPV-94 in MPTP-Induced PD Model Mice

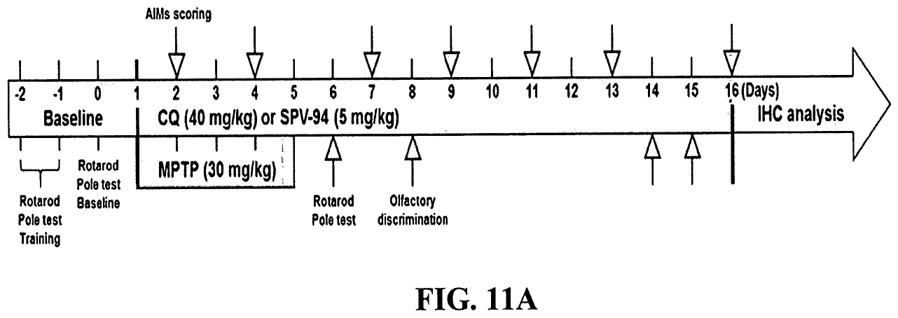

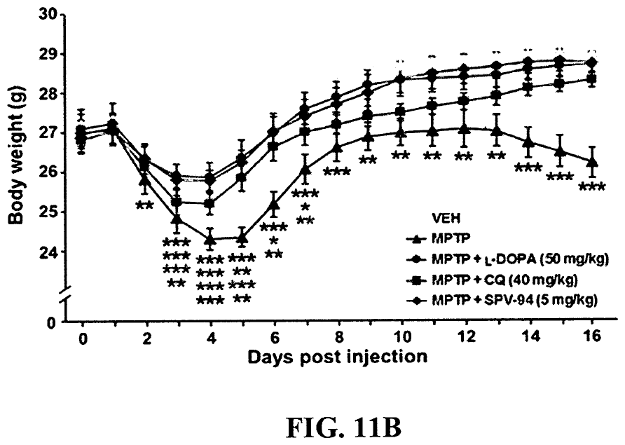

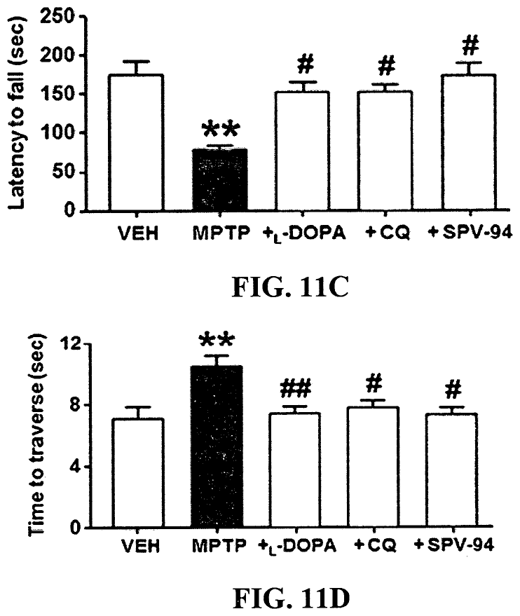

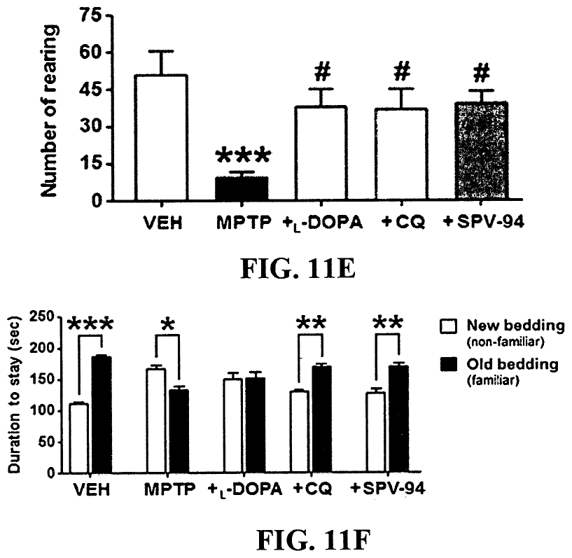

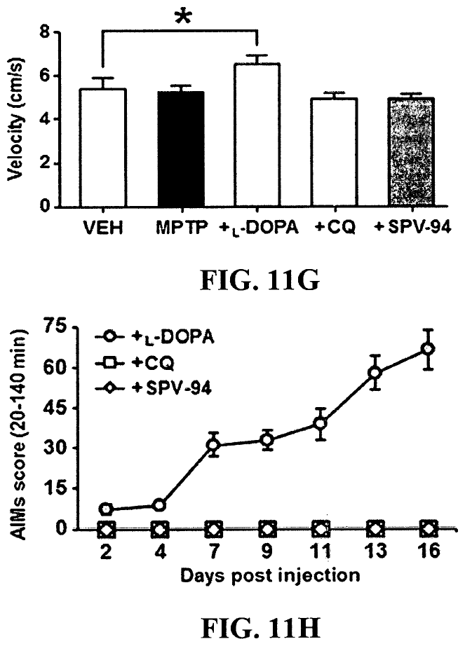

The neuroprotective and immune suppressive effects of CQ and SPV-94 were analyzed in vivo using sub-chronic MPTP-induced (30 mg/kg/day, 5 days) mice model of PD. CQ (40 mg/kg/day) or SPV-94 (5 mg/kg/day) administrations were started with MPTP injection and continued for 16 days (FIG. 11A). To monitor whether CQ and SPV-94 cause dyskinesis, L-DOPA (50 mg/kg/day) administration group was included.

Significant body weight loss was observed starting from the second day of MPTP injection and sustained in MPTP group compared to vehicle control group (VEH). L-DOPA, CQ and SPV-94 treated groups also showed body weight loss but recovered after MPTP injection was completed, and notably, SPV-94 promptly regained body weight right after the last MPTP injection (FIG. 11B).

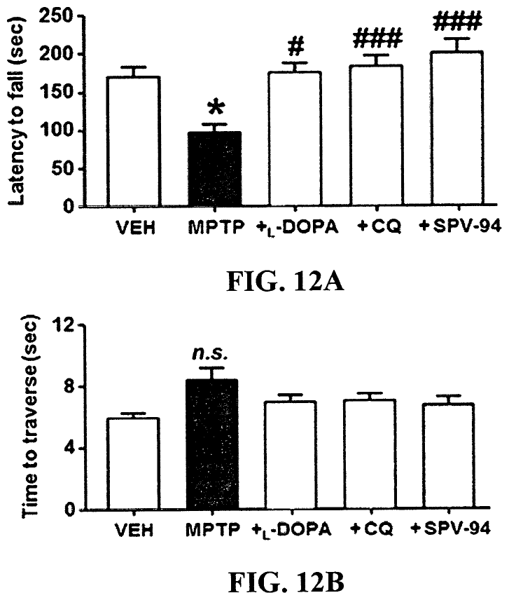

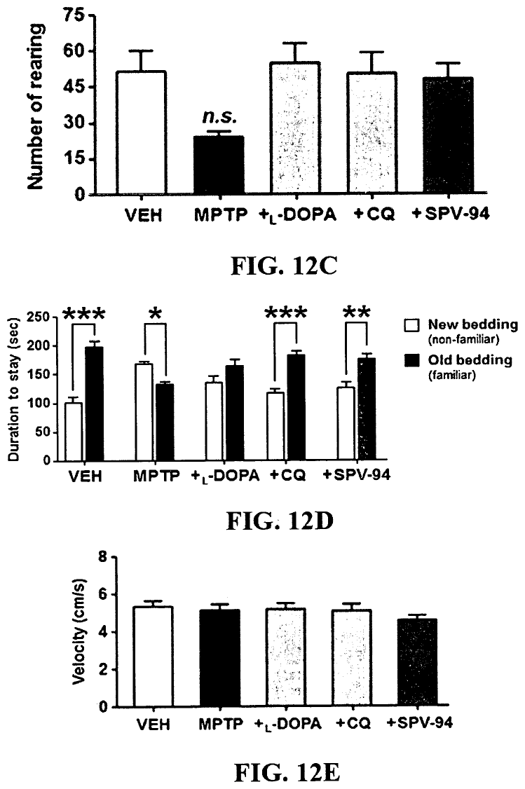

Remarkably, both CQ and SPV-94 administration significantly improved MPTP-induced motor deficits including motor coordination and spontaneous movement assessed using the rotarod, pole test and cylinder test (FIG. 11C-11E). These effects were similar in L-DOPA administration. MPTP-induced motor deficit was maintained until the chronic stage in the rotarod test and treatments of L-DOPA, CQ and SPV-94 led to significant improvement (FIG. 12A), but it was diminished at the chronic stage in the pole test and cylinder test (FIG. 12B and 12C).

We also tested the effects of CQ and SPV-94 in terms of recovery of non-motor symptom such as olfactory dysfunction preceding the motor symptoms in PD (Hawkes et al., J Neurol Neurosurg Psychiatry 1997, 62, 436-446; Braak et al., Cell Tissue Res 2004, 318, 121-134; Chaudhuri et al., Lancet Neurol 2006, 5, 235-245). In the olfactory discrimination test, MPTP-induced mice stayed less time in the old bedding implying failure of distinguish between familiar and non-familiar odor, and L-DOPA could not recover olfaction even with chronic treatment. Interestingly, both CQ and SPV-94 significantly restored olfaction from the sub-acute stage (FIG. 11F and 12D). MPTP injection did not affect the mobility of the mice, but L-DOPA treated mice showed hyperactivity at the sub-acute stage (FIG. 11G).

Next CQ and SPV-94 were compared with L-DOPA in respect of triggering dyskinetic behavior. Mice were measured abnormal involuntary movements (AIMs) scores including axial, limb, and orolingual dyskinesis, every other or third day to monitor AIMs development. Mice received L-DOPA exhibited severe AIMs from 7 days post injection (FIG. 11H). In contrast, neither CQ nor SPV-94 administrations did not develop detectable AIMs throughout the whole monitoring period.

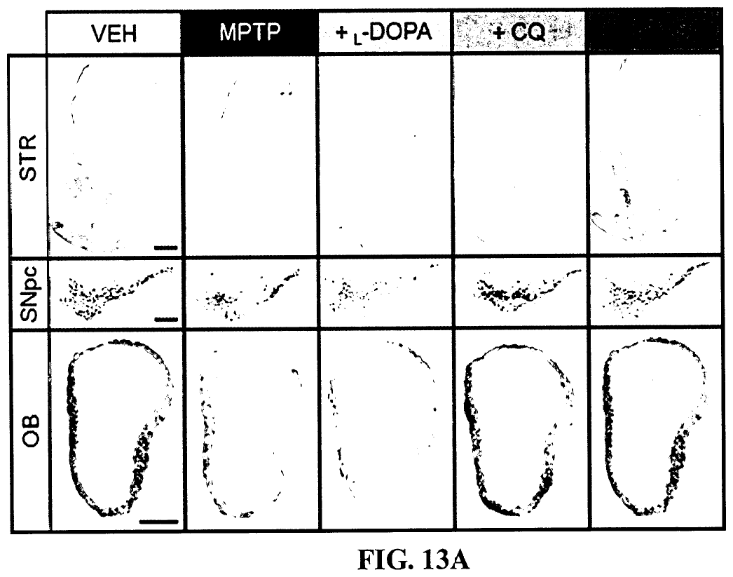

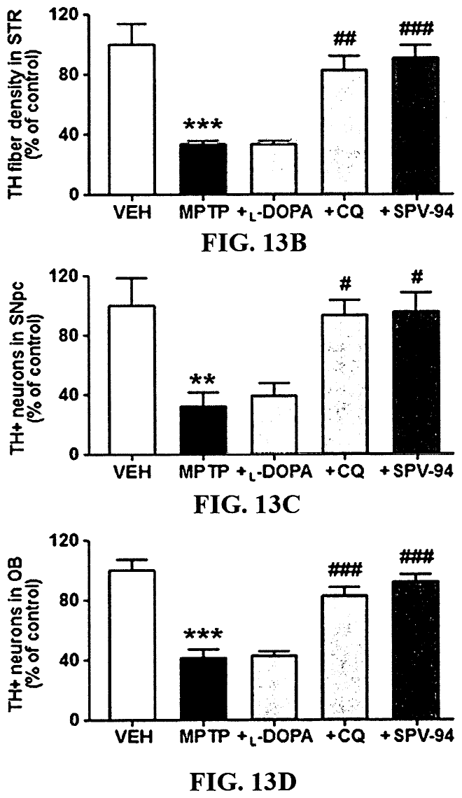

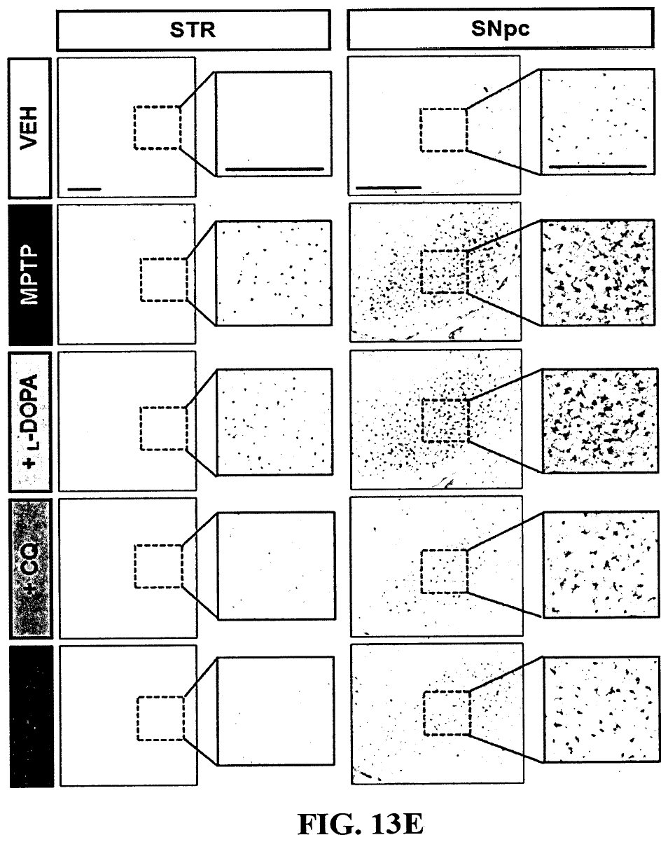

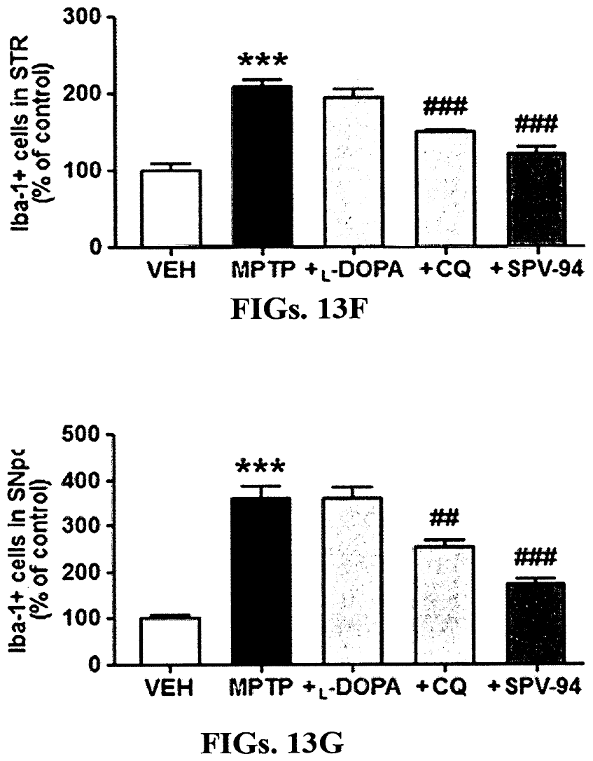

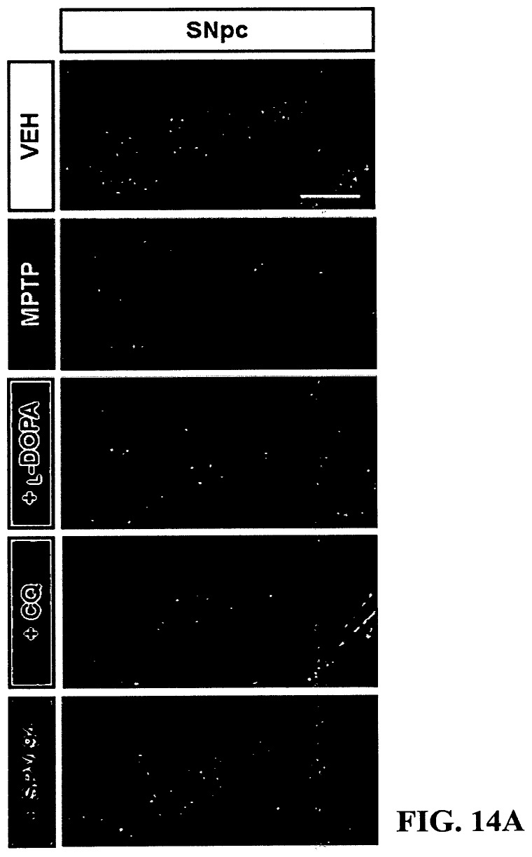

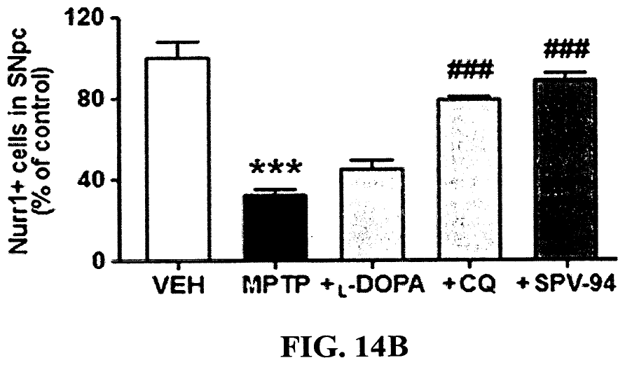

Finally, immunohistochemical analyses revealed that TH⁺ neurons were significantly retained by CQ and SPV-94 but not by L-DOPA in the striatum (STR) and substantia nigra pars compacta (SNpc) regions compared to MPTP treated group (FIGS. 13A-13C). Nun-I expression in the SNpc was corresponded to the TH expression, showing significant decrease in MPTP-treated group and maintained in CQ- or SPV-94-treated groups (FIGS. 14A and 14B).

TH expression was significantly reduced by MPTP treatment also in the olfactory bulb (OB) as corresponding to the previous observations in the MPTP-induced PD models (Prediger et al., Neurotox Res 2010, 17:114-129; Yang et al., Neurotoxicol 2019, 73:175-182; Chen et al., Acta Pharmacol Sin 2019, 40:991-998). Notably, CQ and SPV-94 but not L-DOPA treatments did maintain TH expression in the OB (FIG. 13A and 13D).

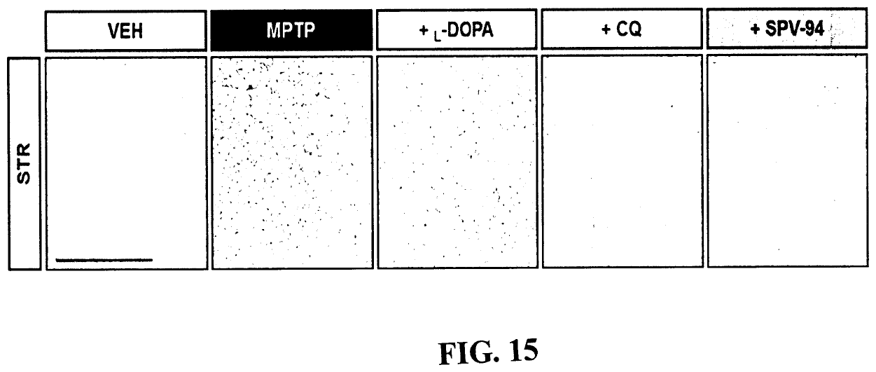

To confirm the immune suppressive function by CQ and SPV-94 in vivo, we detected and quantified immunoreactivity of ionized calcium binding adaptor molecule 1 (Iba-1) as a microglia marker. As shown in FIGS. 13E-13G, MPTP treatment induced significant increase of Iba-1⁺ microglia both in the STR and SNpc. As its immune suppressive function, CQ and SPV-94 markedly reduced Iba-1³⁰ microglia compared to MPTP-treated group. Meanwhile, L-DOPA did not result in reduction of Iba-1 immunoreactivity both in the STR and SNpc (FIGS. 13E-13G). Additional analysis using glial fibrillary acidic protein (GFAP) for activated astrocytes also revealed that increased number of GFAP cells in the STR by MPTP treatment significantly reduced by CQ and SPV-94 but not by L-DOPA (FIG. 15). The data shows that SPV-94 successfully rescue behavioral and pathophysiological deficits involved in PD, making this compound a therapeutic drug for PD.

### Example 6

### MTD (Maximal Tolerated Dose) Comparison Between Compound of Formula (II) and Comparative Example

The objective of this study was to evaluate the escalating dose maximum tolerated dose study of compound of Formula (II) and comparative example

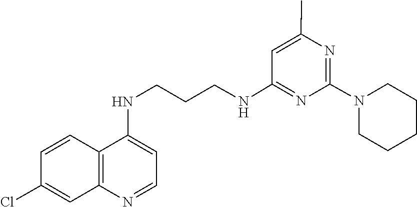 [SMILES: Cc1cc(NCCCNc2ccnc3cc(Cl)ccc23)nc(N2CCCCC2)n1]

in CD-1 mice. Animals were monitored on a daily basis with body weights and clinical signs, and any abnormal observation findings were recorded.

During this study, parts of animals treated with comparative example were found with weight loss, roach back, rough coat, low temperature, low activity, tremble or/and dying by cage side observation. All the animals treated with comparative example at dose level of 40 mg/kg were found with prone, slightly low temperature and twitched to death on the fourth dosing day and no abnormalities were observed in control and animals treated with compound of Formula (II).

Based on these observations, it can be concluded that the test compound of comparative example at 40 mg/kg dose level cannot be tolerated well in CD-1 male mice by oral administration under the current experimental conditions.

| | | Initial | | | | |
|---|---|---|---|---|---|---|
| | | Dose | Dose | | | |
| | | Level | Volume | | | |
| Group | Compound | (mg/kg) | (mL/kg) | Route | Frequency | Number |
| 1 | Saline | — | — | Oral | QD*9 | 2/2 |
| 5 | Formula (II) | 5 | 10 | Oral | QD*9 | 2/2 |
| 7 | Comp. ex. | 5 | 10 | Oral | QD*4 | 2/2 |

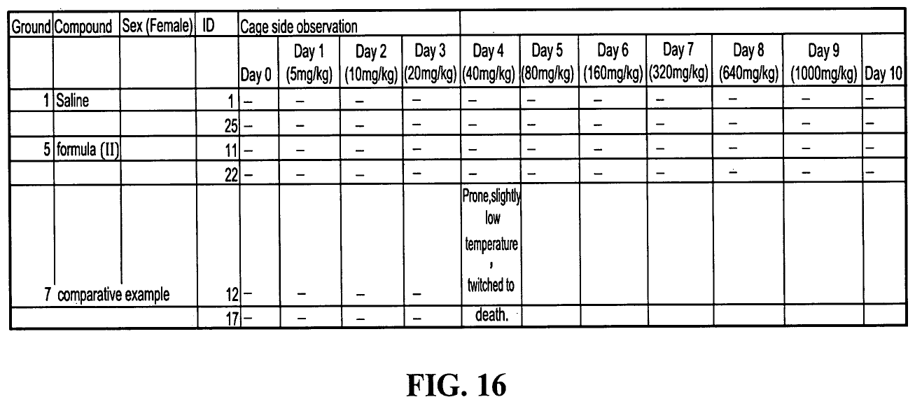

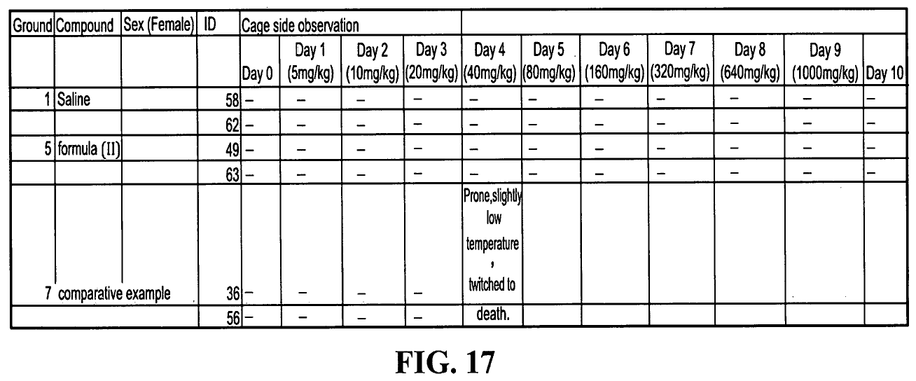

Additional data is provided in FIGS. 16 and 17.

### Example 7

### Pharmacokinetic Data Comparison Between Compound of Formula (II) and Comparative Examples 2 and 3

The objective of this study was to characterize the pharmacokinetics (PK) of compound of Formula (II) in Male SD Rats after single intravenous (IV) and oral (PO) administration. Following PO administration with compound of Formula (II) at dose level of 20 mg/kg, an AUClast of 7341.19 ng/mL was observed. Following PO administration with comparative example 2:

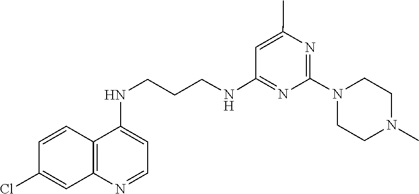 [SMILES: Cc1cc(NCCCNc2ccnc3cc(Cl)ccc23)nc(N2CCN(C)CC2)n1]

at dose level of 20 mg/kg, an AUClast of 250.06 ng/mL was observed. Following PO administration with comparative example 3:

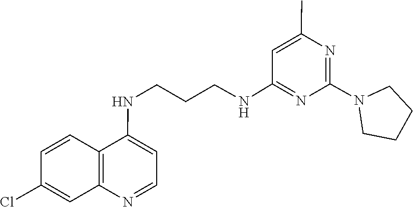 [SMILES: Cc1cc(NCCCNc2ccnc3cc(Cl)ccc23)nc(N2CCCC2)n1]

at dose level of 20 mg/kg, an AUClast of 270.71 ng/mL was observed.

Pharmacokinetic Study
Compound: Formula (II)
Dosing route: Oral
Dose: 20 mg/kg
Animal: Rat

| Time (h) | Plasma Conc. (ng/mL) |
|---|---|
| 0.083 | 10.70 |
| 0.25 | 118.43 |
| 0.5 | 559.00 |
| 1 | 1175.67 |
| 2 | 1020.00 |
| 4 | 952.67 |
| 8 | 294.33 |
| 24 | 16.83 |
| AUClast (h*ng/mL) | 7341.19 |

Pharmacokinetic Study
Compound: comp. ex. 2
Dosing route: Oral
Dose: 20 mg/kg
Animal: Rat

| Time (h) | Plasma Conc (ng/mL) |
|---|---|
| 0.083 | 5.15 |
| 0.25 | 6.68 |
| 0.5 | 9.91 |
| 1 | 13.87 |
| 2 | 14.93 |
| 4 | 12.00 |
| 8 | 16.53 |
| 24 | 5.92 |
| AUClast (h*ng/mL) | 250.06 |

Pharmacokinetic Study
Compound: comp. ex. 3
Dosing route: Oral
Dose: 20 mg/kg
Animal: Rat

| Time (h) | Plasma Conc (ng/mL) |
|---|---|
| 0.083 | 7.71 |
| 0.25 | 16.02 |
| 0.5 | 16.58 |
| 1 | 19.45 |
| 2 | 23.27 |
| 4 | 19.50 |
| 8 | 14.50 |
| 24 | 3.53 |
| AUClast (h*ng/mL) | 270.71 |

### OTHER EMBODIMENTS

It is to be understood that while the present application has been described in conjunction with the detailed description thereof, the foregoing description is intended to illustrate and not limit the scope of the present application, which is defined by the 5 scope of the appended claims. Other aspects, advantages, and modifications are within the scope of the following claims.

1. A compound selected from any one of the following compounds: 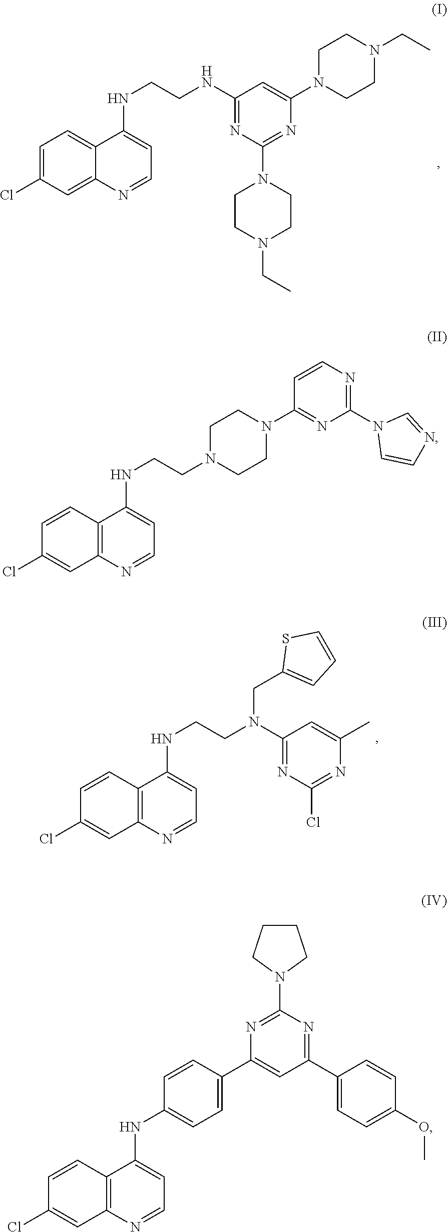 [SMILES: CCN1CCN(c2cc(NCCNc3ccnc4cc(Cl)ccc34)nc(N3CCN(CC)CC3)n2)CC1 ; Clc1ccc2c(NCCN3CCN(c4ccnc(-n5ccnc5)n4)CC3)ccnc2c1 ; Cc1cc(N(CCNc2ccnc3cc(Cl)ccc23)Cc2cccs2)nc(Cl)n1 ; COc1ccc(-c2cc(-c3ccc(Nc4ccnc5cc(Cl)ccc45)cc3)nc(N3CCCC3)n2)cc1] 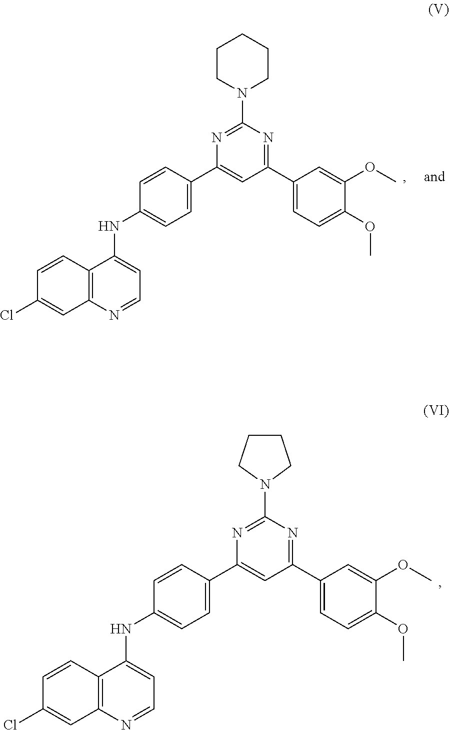 [SMILES: COc1ccc(-c2cc(-c3ccc(Nc4ccnc5cc(Cl)ccc45)cc3)nc(N3CCCCC3)n2)cc1OC ; COc1ccc(-c2cc(-c3ccc(Nc4ccnc5cc(Cl)ccc45)cc3)nc(N3CCCC3)n2)cc1OC]
or a pharmaceutically acceptable salt thereof.

2. The compound of claim 1, having Formula (I): 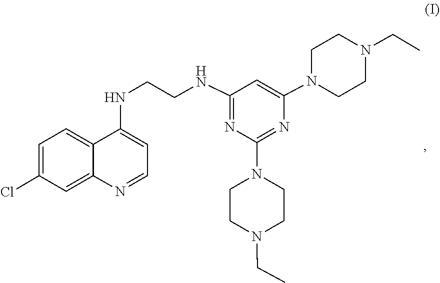 [SMILES: CCN1CCN(c2cc(NCCNc3ccnc4cc(Cl)ccc34)nc(N3CCN(CC)CC3)n2)CC1]
or a pharmaceutically acceptable salt thereof.

3. The compound of claim 1, having Formula (II): 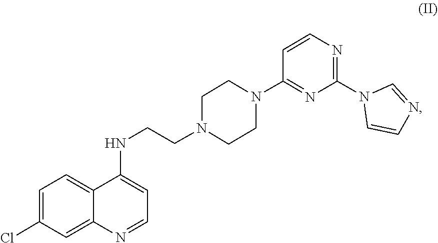 [SMILES: Clc1ccc2c(NCCN3CCN(c4ccnc(-n5ccnc5)n4)CC3)ccnc2c1]
or a pharmaceutically acceptable salt thereof.

4. The compound of claim 1, having Formula (III): 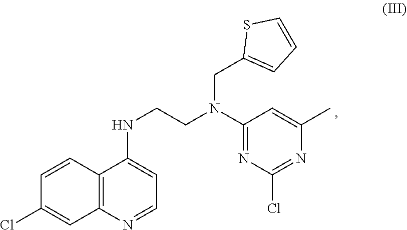 [SMILES: Cc1cc(N(CCNc2ccnc3cc(Cl)ccc23)Cc2cccs2)nc(Cl)n1]
or a pharmaceutically acceptable salt thereof.

5. The compound of claim 1, having Formula (IV): 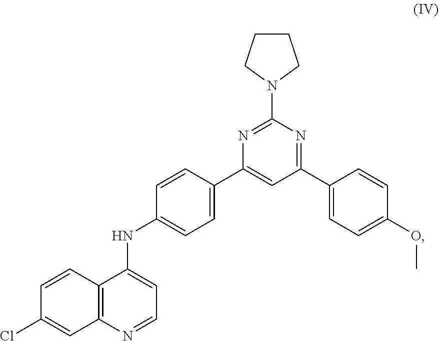 [SMILES: COc1ccc(-c2cc(-c3ccc(Nc4ccnc5cc(Cl)ccc45)cc3)nc(N3CCCC3)n2)cc1]
or a pharmaceutically acceptable salt thereof.

6. The compound of claim 1, having Formula (IV): 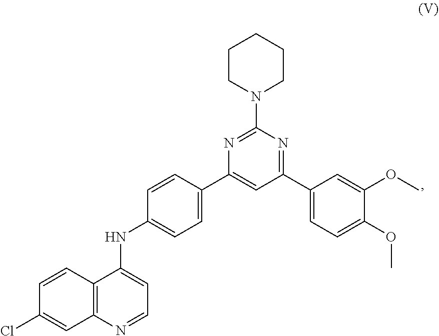 [SMILES: COc1ccc(-c2cc(-c3ccc(Nc4ccnc5cc(Cl)ccc45)cc3)nc(N3CCCCC3)n2)cc1OC]
or a pharmaceutically acceptable salt thereof.

7. The compound of claim 1, having Formula (IV): 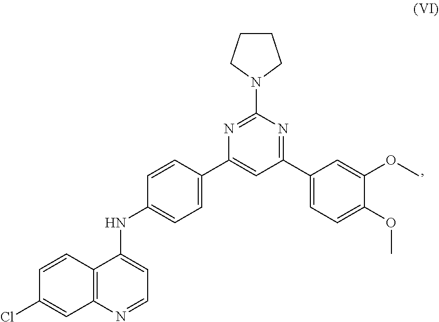 [SMILES: COc1ccc(-c2cc(-c3ccc(Nc4ccnc5cc(Cl)ccc45)cc3)nc(N3CCCC3)n2)cc1OC]
or a pharmaceutically acceptable salt thereof.

8. A pharmaceutical composition comprising a compound of claim 1, or a pharmaceutically acceptable salt thereof, and a pharmaceutically acceptable carrier.

9. A method of modulating Nurr1 activity in a cell, the method comprising contacting the cell with an effective amount of a compound of claim 1, or a pharmaceutically acceptable salt thereof.

10. The method of claim 9, wherein the modulating of the Nurr1 activity comprises increasing the Nurr1 activity in the cell.

11. The method of claim 9, comprising contacting the cell in vivo, in vitro, or ex vivo.

12-15. (canceled)

16. A method of treating a disease or condition in which decreased Nurr1 activity or Nurr1 hypoactivity contributes to the pathology or symptomology of the disease, the method comprising administering to the subject a therapeutically effective amount of a compound of claim 1.

17. The method of claim 16, wherein the disease or condition is a neurodegenerative disease.

18. The method of claim 17, wherein the neurodegenerative disease is Parkinson's disease.

19. The method of claim 17, wherein the neurodegenerative disease is Alzheimer's disease.

20. (canceled)

21. The method of claim 16, wherein the disease or condition is inflammation or inflammation-associated disease or condition.

22. (canceled)

23. A method of treating an infectious disease or disorder, the method comprising administering to the subject a therapeutically effective amount of a compound of claim 1, or a pharmaceutically acceptable salt thereof.

24. The method of claim 23, wherein the infectious disease is malaria.

25. (canceled)

26. A method of inducing differentiation of a stem cell into a dopaminergic neuron, the method comprising contacting the stem cell with a compound of claim 1, or a pharmaceutically acceptable salt thereof.

27. The method of claim 26, wherein the stem cell is a human embryonic stem cell.
